(19) United States
(12) Patent Application Publication
(10) Pub. No.: US 2010/0317677 A1
(43) Pub. Date: Dec. 16, 2010

(54) Methods of Treating a Microbial Infection by Modulating RNase-L Expression and/or Activity

(76) Inventors: Bret A. Hassel, Woodbine, MD (US); Alan S. Cross, Chevy Chase, MD (US); Xiao-Ling Li, Columbia, MD (US); Tae Jin Kang, Seoul (KR)

Correspondence Address: MORGAN LEWIS & BOCKIUS LLP, 1111 PENNSYLVANIA AVENUE NW, WASHINGTON, DC, 20004, US

(21) Appl. No.: 12/677,692

(22) PCT Filed: Sep. 10, 2008
(86) PCT No.: PCT/US08/75767

§ 371 Date: Aug. 20, 2010

Related U.S. Application Data
(60) Provisional application No. 60/971,367, filed on Sep. 11, 2007.

(51) Int. Cl. A61K 31/519; A61K 31/381; A61K 31/426; A61K 31/4162; A61K 31/17; A61K 31/24; A61P 31/04

(52) U.S. Cl. 514/267; 514/445; 514/369; 514/406; 514/586; 514/535; 514/537

(57) ABSTRACT

The invention relates to methods and compositions for treating a microbial infection. In the present invention, RNase-L activity has been shown to play an integral role in innate immunity and for defense against invading microbes. The present invention is drawn to exploiting the role of RNase-L in innate immunity for methods of treating a microbial infection. The present invention is also drawn to exploiting the role of RNase-L in innate immunity for methods of treating an immune related disease or disorder.

### CROSS-REFERENCE TO RELATED APPLICATIONS

The application claims priority to U.S. Provisional Application No. 60/971,367, filed 11 Sep. 2007, which is incorporated by reference in its entirety.

### STATEMENT REGARDING FEDERALLY SPONSORED RESEARCH OR DEVELOPMENT

This invention was made with government support under NIH Grant No. U54 AI057168-01 awarded by the National Insitutes of Health. The government has certain rights in the invention.

### BACKGROUND OF THE INVENTION

1. Field of the Invention

The invention relates to innate immunity. The invention further relates to methods of treating a microbial infection.

2. Background of the Invention

Type 1 interferons as essential mediators of the host immune response

Type 1 interferons (IFNs) were discovered fifty years ago as the primary antiviral cytokines. However, their function in the innate immune response to nonviral pathogens has only recently gained recognition (Stetson et al. (2006) Immunity 25, 373-381; Decker et al. (2002) The Journal of clinical investigation 109, 1271-1277). The elucidation of Toll-Like Receptor (TLR) and non-TLR signaling pathways that function to detect microbial infection and activate expression of host innate immune genes revealed that the induction of type 1 IFNs was a central component of the genetic response to both viral and bacterial pathogens (Akira, S. (2006) Current topics in microbiology and immunology 311, 1-16; Mariathasan et al. (2007) Nature reviews 7, 31-40). Specifically, viral and bacterial nucleic acid TLR agonists, and LPS from gram-negative bacteria, all activate an overlapping signal transduction pathway that converges on IFN-regulatory factor-3 (IRF3), a transcription factor that is required for IFN-beta induction. Importantly, the induction of IFN by bacteria, or bacterial TLR agonists, is required for the successful resolution of infections by a diverse profile of bacteria, demonstrating its functional role in host defense from bacterial challenge (Decker et al. (2002) The Journal of clinical investigation 109, 1271-1277; Karaghiosoff et al. (2003) Nature immunology 4, 471-477). Thus, a current challenge is to determine the molecular mechanisms by which IFNs exert their antibacterial activity.

The broader role for type1 IFNs in the innate immune response to viral and bacterial pathogens suggested that common downstream effectors are involved. Specifically, established mediators of IFN antiviral action may serve previously unrecognized roles in antibacterial immunity. The induction of IFN gene expression by microbial infection results in its secretion from cells where it acts in an autocrine or paracrine manner to modulate the activities of effector cells (e.g., natural killer cells and cytotoxic T-lymphocytes) and to activate a gene expression program that results in enhanced cellular antimicrobial activities (Stetson et al. (2006) Immunity 25, 373-381). The binding of IFN to its receptor activates the janus kinase/signal transducer and activator of transcription (JAK/STAT) signaling pathway which culminates in the transcriptional induction of IFN-stimulated genes (ISGs; Stark et al. (1998) Annu Rev Biochem 67, 227-264). Although the full spectrum of activities mediated by the ˜400 ISGs remains to be determined, several ISGs serve well established functions in IFN action. RNase-L and PKR, two of the best studied ISGs that were originally identified based on their antiviral activities, have more recently been shown to function in IFN-dependent and IFN-independent, antiproliferative/tumor suppressive activities (Carpten et al. (2002) Nat Genet 30, 181-184; Meurs et al. (1993) Proc Natl Acad Sci USA 90, 232-236.), demonstrating the capacity of these effectors to mediate pleiotropic functions.

RNase-L

RNase-L is the terminal component of an IFN-regulated RNA decay pathway (FIG. 1) that was discovered as a mediator of host antiviral activity, and was subsequently determined to function in apoptosis, senescence, and tumor suppression as an endogenous constraint on cell growth (Silverman, R. H. (2003) Biochemistry 42, 1805-1812.). Most cell types express a low, basal level of RNase-L that is inactive in the absence of its allosteric activator, 2′,5′-linked ligoadenylate, 2-5A (pppA(2′p5′A)n n>2). 2-5A is produced by a family of 2′,5′oligoadenylate synthetases (OAS) that are induced by IFN and microbial challenge, and require double stranded RNA (dsRNA) as a cofactor for enzymatic activity. 2-5A binding induces the dimerization and enzymatic activation of RNase-L resulting in the endonucleolytic cleavage of single stranded RNA with a preference for UpN sequences (Wreschner et al. (1981) Nature 289, 414-417). RNase-L activity is attenuated by cellular phosphatases and a 2′phosphodiesterase that inactivate 2-5A, and by a protein inhibitor of RNase-L, RLI (Benoit De Coignac et al. (1998) Gene 209, 149-156; Kubota et al. (2004), J Biol Chem 279, 37832-37841).

Cellular mRNAs and rRNAs, and viral RNAs, have been identified as RNase-L substrates (Li et al (1998) J Virol 72, 2752-2759; Bisbal et al. (2000) Mol Cell Biol 20, 4959-4969; Chandrasekaran et al. (2004) Biochem Biophys Res Commun 325, 18-23; Khabar et al. (2003) J Biol Chem 278, 20124-20132); however, the precise molecular mechanisms remain to be determined. For example, in the context of viral infection the degradation of viral RNAs by RNase-L is clearly an important component of its antiviral activity, however, the potential contribution of RNase-L-dependent regulation of host genes to the innate immune response has not been examined. In this regard, the RNase-L-dependent induction of apoptosis is observed as an antiviral strategy and as a stress response, independent of viral infection, suggesting that it involves the RNase-L activation by endogenous 2-5A, and regulation of as of yet unidentified host mRNAs (Castelli et al. (1997) J Exp Med 186, 967-972; Diaz-Guerra et al. (1997) Virology 236, 354-363). Furthermore, two studies implicated RNase-L in the host immune response in the absence of direct microbial targets of RNase-L action (e.g., pathogen-derived RNAs), strengthening the notion that this activity is mediated through the regulation of host transcripts. Specifically, RNase-L−/− mice exhibited significantly reduced antigenicity to a DNA vaccine antigen (Leitner et al. (2003) Nat Med 9, 33-39), and displayed a delayed rejection of MHC class II disparate skin allografts (Silverman et al. (2002) Viral immunology 15, 77-83). In the former example, dsRNA that is produced in the course of immunization with the alphavirus replicon and was proposed to activate OAS resulting in the production of 2-5A, however, the mechanisms by which RNase-L impacted the immune response, and the host genes involved, are not known. In an effort to determine the role of RNase-L-dependent regulation in host gene expression, microarray analyses were performed following RNase-L activation by 2-5A transfection ((Malathi et al. (2005) Proc Natl Acad Sci USA 102, 14533-14538) and Hassel, unpublished). These studies revealed that a finite number of transcripts exhibited RNase-L-dependent regulation. Moreover, both downregulated mRNAs that represent candidate RNase-L substrates and upregulated transcripts that represent the indirect effects of RNase-L action (e.g., if a RNase-L substrate encodes a transcriptional repressor) were identified. Importantly, these findings indicate that RNase-L activation does not result in a global increase in RNA turnover, but, rather, it has the capacity to selectively target specific RNAs for degradation.

Cathepsin E and Endolysosomal Activities

Cathepsin-E (catE) is an aspartic proteinase of the pepsin superfamily that is expressed primarily in immune cells including antigen presenting cells (APCs), lymphoid tissues, and gastric epithelium (Yanagawa et al. (2007) J Biol Chem 282, 1851-1862; Yasuda et al. (2005) J Biochem (Tokyo) 138, 621-630). Several transcription factors contribute to the tissue specific expression of catE. Specifically, PU.1, GATA1, AP1, and p300 all enhance catE transcription, whereas YY1 and the type III isoform of class II transactivator repress transcription (Cook et al. (2001) Eur J Biochem 268, 2658-2668; Yee et al. (2004) J Immunol 172, 5528-5534). In addition to these transcriptional constraints on the tissue distribution of catE expression, our preliminary studies demonstrated that, RNase-L functions to maintain low basal levels of catE expression in macrophages through the regulation of its mRNA half-life. This is the first evidence of the post-transcriptional regulation of catE expression, and provides a mechanism to rapidly modulate catE expression, possibly through the upstream regulation of RNase-L, in response to immune or microbial stimuli. Indeed, catE mRNA is induced in response to IFN-gamma, and is repressed by IL-4, indicating that catE expression is responsive to immunomodulatory cytokines (Tsukuba et al. (2003) J Biochem (Tokyo) 134, 893-902). However, it is not known if the cytokine-induced modulation of catE expression occurs through transcriptional or post-transcriptional mechanisms.

Consistent with its restricted tissue distribution in immune cells, catE is implicated in a broad spectrum of physiological and pathophysiological activities that are associated with immune functions (Yanagawa et al. (2007) J Biol Chem 282, 1851-1862; Tsukuba et al. (2003) J Biochem (Tokyo) 134, 893-902; Nishioku et al. (2002) J Biol Chem 277, 4816-4822; Chain et al. (2005) J Immunol 174, 1791-1800; Tsukuba et al. (2006) J Biochem (Tokyo) 140, 57-66). Evidence of a role for catE in antimicrobial immunity was first provided by the finding that catE−/− mice that develop atopic dermatitis when reared in conventional, but not pathogen-free, conditions (Tsukuba et al. (2003) J Biochem (Tokyo) 134, 893-902). Subsequent studies demonstrated an increased mortality of catE−/− mice following challenge with gram-positive and -negative bacteria, confirming a role for catE in host antibacterial defense (Tsukuba et al. (2006) J Biochem (Tokyo) 140, 57-66). CatE is localized primarily in endolysosomal compartments that serve critical functions in the elimination of microbial pathogens by phagocytic cells (Blander et al. (2006) Nature immunology 7, 1029-1035). Internalization of microbes induces the bacteriocidal action of reactive oxygen species, and subsequent phagosome maturation results in the transfer of cargo to increasingly acidified endocytic compartments in which proteases with low pH optima hydrolyze their contents (Aderem et al. (1999) Annual review of immunology 17, 593-623). The endolysosomal localization of catE suggests that it mediates immune functions through the regulation of this pathway. Consistent with this idea, catE−/− macrophages exhibited an elevated lysosome pH, and increased expression of the major lysosomal membrane proteins, LAMPs 1 and 2 (LAMP 1/2), that were recently identified as catE substrates (Yanagawa et al. (2007) J Biol Chem 282, 1851-1862). LAMP 1/2 proteins are required for the maturation of phagosomes and the delivery of cargo to lysosomes (Eskelinen, E. L. (2006) Molecular aspects of medicine 27, 495-502; Huynh et al. (2007) Embo J 26, 313-324). In addition, cells from LAMP 1/2-deficient mice exhibit defects in autophagy, a process that eliminates internal, rather than phagocytosed, cellular and microbial cargo via the endolysosomal pathway, and serves critical functions in antimicrobial immunity and antigen presentation (Kirkegaard et al (2004) Nat Rev Microbiol 2, 301-314; Menendez-Benito et al. (2007) Immunity 26, 1-3). Taken together, these studies indicate that dysregulated LAMP 1/2 expression, either upregulated as in catE−/− macrophages or downregulated as in LAMP1/2−/− cells, results in impaired lysosomal activity which may underlie associated defects in immune function. In agreement with this, inhibition of catE activity blocks MHC class II antigen presentation that, in turn, is dependent on endolysosomal functions for processing of the invariant chain and its association with exogenous and endogenous peptides in late endosomes (Nishioku et al. (2002) J Biol Chem 277, 4816-4822; Chain et al. (2005) J Immunol 174, 1791-1800; Menendez-Benito et al. (2007) Immunity 26, 1-3). Thus, the catE-mediated regulation of LAMP 1/2 and lysosomal function provides a mechanistic basis for its role in MHC II presentation.

The observation that catE−/− mice exhibited compromised induction of proinflammatory cytokines in response to TLR agonists provided a second potential link between catE-associated immune activities and its regulation of endolysosomal function (Tsukuba et al. (2006) J Biochem (Tokyo) 140, 57-66). Specifically, recent studies demonstrated that a functional TLR signaling pathway is required for the maturation of phagosomes containing microbial cargo (Blander et al. (2006) Nature immunology 7, 1029-1035; Blander et al. (2004) Science 304, 1014-1018). However, the reciprocal relationship (i.e., that components of the endolysosomal system may be required for optimal TLR signaling following microbe internalization), has not been examined. In this scenario, the impaired lysosome function observed in catE−/− macrophages may be linked to the diminished induction of proinflammatory cytokines in these cells. Thus, the regulation of LAMP 1/2 proteins and lysosome function by catE may account for a significant component of its immunomodulatory activities.

Microbial Defense

Mammals have evolved potent, multidimensional strategies to combat microbial pathogens and effectively resolve infections in healthy individuals. However, successful microbes can counter these strategies by evading or subverting components of the host immune response resulting in disease and mortality. In light of this, microbial infections remain major causes of morbidity and mortality around the world. Current antimicrobial therapy is increasingly compromised by the emergence and spread of microbes resistant to commonly used antimicrobial agents. This resistance is due largely to the substantial quantities of antibiotics that are administered in health care, and even non-health care settings. Empiric use of antimicrobial agents for questionable infections, spectra of therapy that are more broad than are indicated by likely pathogens, prolonged therapy after successful treatment and widespread use of antibiotics in food industries all contribute in significant ways to the growing problem of resistance. Thus, novel methods of treating microbial infection represents a long-felt need in the art. To this end, the invention disclosed herein relates to novel methods of treating microbial infection.

### SUMMARY OF THE INVENTION

The invention relates to agents that regulate innate immunity, compositions comprising the same, and methods of treatment comprising administering the same.

In certain embodiments, the invention is drawn to a method of treating a microbial infection in a subject in need thereof comprising administering an agent that increases the activity of RNase-L. In other embodiments, the invention is drawn to a method of treating a subject at risk of suffering from a microbial infection comprising administering an agent that increases the activity of RNase-L.

In certain embodiments, the agent is selected from the group consisting of a nucleic acid, a small molecule, and any combination thereof. In specific embodiments, the nucleic acid is selected from the group consisting of a nucleic acid comprising cathepsinE (catE) mRNA and a 2′,5′-linked oligoadenylate (2-5A). In other specific embodiments, the small molecule is selected from the group consisting of C-5966451, C-5950331, C-5972155, C-5947495, C-6131864, C-6131645, C-6131416, C-6645744, C-6474572, C-5142087, and C-5973265.

In certain embodiments, the agent is administered prior to, concurrently with, or following the administration of one or more therapies used to treat a microbial infection. In other embodiments, the one or more therapies used to treat a microbial infection is selected from the group consisting of a bacterial therapy or a viral therapy.

In certain embodiments, the invention is drawn to a method of treating a microbial infection in a subject in need thereof comprising administering an agent that increases the expression of RNase-L. In other embodiments, the invention is drawn to a method of treating a subject at risk of suffering from a microbial infection comprising administering an agent that increases the expression of RNase-L. In specific embodiments, the agent that increases the expression of RNase-L comprises a vector comprising a polynucleotide encoding RNase-L or encoding a functional part thereof.

In certain embodiments, treating a microbial infection is the treatment of a bacterial infection. In further embodiments, the bacterial infection is caused by a bacterium selected from the group consisting of methicillin-resistant Staphylococcus aureus (MRSA), Bacillus anthracis (BA) and Escherichia coli (E. coli).

In certain embodiments, the invention is drawn to a method of treating an immune related disease or disorder in a subject in need thereof comprising administering an agent that decreases the activity of RNase-L. In other embodiments, the invention is drawn to a method of treating an immune related disease or disorder in a subject in need thereof comprising administering an agent that decreases the expression of RNase-L.

In certain embodiments, a method of treating an immune related disease or disorder in a subject in need thereof comprising administering an agent that decreases the activity or expression of RNase-L is administered prior to, concurrently with, or following the administration of one or more immune modulating molecules.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF THE DRAWINGS

FIG. 1. 2-5A pathway. dsRNA: double-stranded RNA; ssRNA: single-stranded RNA; 2′-PDE: 2′-phosphodiesterase; p′tase: phosphatase.

FIG. 2. Increased susceptibility of RNase-L−/− mice to bacterial challenge. (A) and (B) RNase-L−/− and WT mice (7 mice per group) were challenged with BA spores or E. coli at the indicated doses; survival was monitored and Kaplan Meier analyses are shown (Kaplan E L, M. P. (1958) Journal of the American Statistical Association 53, 457-481). (C) Peritoneal macrophages from RNase-L−/− and WT mice were infected with BA spores (MOI=0.2) for the indicated times, then the viable spores were quantified, and expressed as the log reduction in CFU as compared to the 1 hour value.

FIG. 3. RNase-L deficient mice are unable to resolve infection by E. coli. (A) Microbial titres. Organs, peritoneal lavage fluid, and blood were collected from E. coli infected mice (2.5×103 cfu) and values at 0 and 72 hrs post-infection are shown. Organ titres are presented as cfu/g and peritoneal fluid and blood are displayed as cfu/ml. (B) Plasma IL-1β and TNFα were measured by ELISA at the indicated times post-infection, values are the average for three mice. (C) Macrophages, Neutrophils, and Lymphocytes in peritoneal fluid are expressed as a percentage of ˜1600 cells counted from four mice; the small percentage of cells that did not fall into these categories (e.g. eosinophils) are not shown.

FIG. 4. RNase-L-dependent gene expression in BA-infected macrophages. (A) Immune response genes that exhibited >1.75-fold difference in induction at 8 hpi with BA spores (MOI=1.0) in RNase-L−/− (KO) and WT macrophages. (B) qPCR validation of IL-1β and TNFα mRNA expression (normalized to constitutively expressed GAPDH mRNA).

FIG. 5. RNase-L-dependent regulation of catE in macrophages and in vivo. (A) Steady state expression of catE mRNA in macrophages+/−BA infection (MOI=1.0); values are normalized to constitutively expressed GAPDH mRNA. (B) qPCR quantification of catE mRNA expression in tissues from uninfected mice; tissue number designations refer to the mouse sample used. (C) Western blot of catE protein in RNase-L−/− and WT macrophages; the blot was reacted with constitutively expressed α-actin as a loading control. (D) decay kinetics of catE mRNA following transcriptional arrest by ActD in RNase-L−/− and WT macrophages. (E) Half-life values for stable and unstable mRNAs in RNase-L−/− and WT macrophages.

FIG. 6. Expression of LAMP 1/2 proteins is reduced in RNase-L−/− macrophages. LAMP 1/2, catE, and α-actin proteins in RNase-L−/− and WT macrophages were measured by Western blot.

FIG. 7. Phagocytic activation profile is altered in RNase-L−/− macrophages. At the indicated times following infection with E. coli (2.5×103 cfu), cells in the peritoneal fluid were isolated and stained; representative fields are shown at 200× and 400× (inset) magnification. Cell types are labeled in the RNase-L−/− 72 h field: macrophage (the predominant cell type), #; lymphocyte, *; neutrophil, arrowhead.

FIG. 8. Model in which the RNase-L-dependent regulation of catE is required for LAMP expression and lysosome associated immune functions in macrophages. Solid arrows in macrophage indicate multiple steps in phagosome maturation not shown on diagram.

FIG. 9. RNase-L dependent modulation IL1-b, TNFa, and IFNb induction observed following bacterial infection of mice and macrophages

FIG. 10. Alignment of human and murine catE mRNA orthologues. Numbers refer to the sizes of each region in bp, and an asterix indicates the locations of putative RNase-L recognition elements. Shaded boxes indicate regions of >75% sequence identity as determined by Clustal W alignment.

FIG. 11. CatE exhibits increased expression and protracted association with BA spore components in RNase-L−/− macrophages. A. WT and RNaseL−/− macrophages were uninfected, or infected for the indicated times with Sterne or sporulation deficient, Δ-Ger, strains of BA spore (MOI=5). Cells were fixed and immunostained for spores (green), CatE (red), macrophage marker (CD11b, blue), and nucleic acid (DAPI, white). Arrowheads identify CatE co-localized with BA spores. Spores that have not germinated (4 h, and Δ-Ger panels) exhibit a rounded morphology and fluoresce green (B, middle panel) or orange (in merged panels due to co-localization with CatE). Note that co-localization of CatE with spore components is lost upon germination in WT, but not RNase-L−/− macrophages (compare WT and RNase-L−/− at 6 h and 11B); arrowheads at 5.6 h Sterne in WT macrophages indicate co-localization in rare, ungerminated spores, whereas virtually all immunoreactive spore components co-localize with CatE at these time points in RNase-L−/− macrophages (representative examples indicated by arrowheads). This distinct post-germination pattern of CatE-spore co-localization was reproducibly observed in all fields, and is representative of three independent experiments. B. WT and RNase-L−/− macrophages were BA infected for 5 h and stained as in A, and signals were merged (left panels), or filtered to detect only spores (middle panels) or CatE (right panels) in identical fields. Arrowheads indicate CatE-spore co-localization; WT macrophages in which spores have germinated are outlined to illustrate the diffuse staining and loss of co-localization. C. Differential co-localization of CatE with BA spores in WT and RNase-L−/− (KO) macrophages. Arrowheads indicate colocalization of CatE and BA spores prior to germination, and post-germination in KO but not WT macrophages. The scale bars in all panels=10 μm.

FIG. 12. Bacteria- and TLR agonist-induced signaling and gene expression are diminished in RNase-L−/− macrophages. A. IL1β induction was measured by qRTPCR following LPS stimulation of RAW264.7 macrophages that had been stably transfected with CatE or vector control; expression of transduced CatE is shown in FIG. 12E. B. cytokine expression following treatment of WT and RNase-L−/− macrophages with the TLR3 and 4 agonists, dsRNA and LPS respectively, was measured by qRTPCR. C and D. ERK1/2 and Stat1 phsophorylation, the degradation of IκB, and dimerization of IRF-3 protein were measured following E coli infection. Experiments were conducted in triplicate; IRF-3 dimers were quantified by densitometry. Induction of IFNβ mRNA (lower panel of D) was measured by qRTPCR following E. coli infection. For all bar graphs, error bars are s.d. and (*) signifies p<0. [symbol] 05; (**) signifies p<0.001.

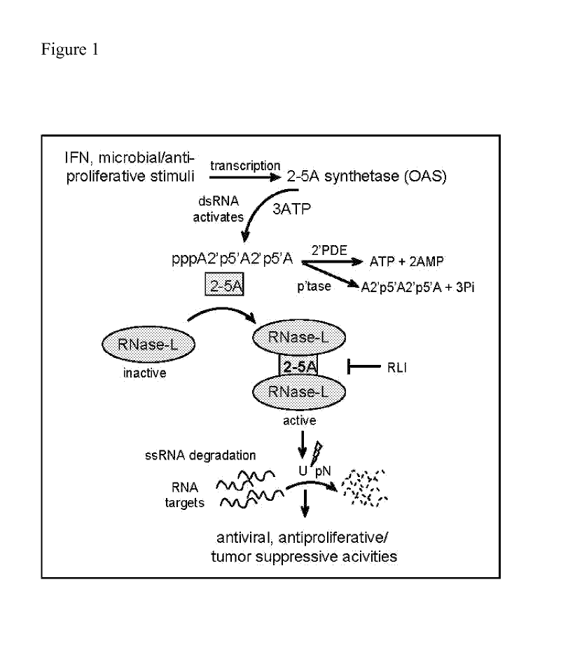

### DETAILED DESCRIPTION OF THE INVENTION

I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein in the claim(s), when used in conjunction with the word “comprising”, the words “a” or “an” may mean one or more than one. As used herein “another” may mean at least a second or more. Furthermore, unless otherwise required by context, singular terms shall include pluralities and plural terms shall include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one would consider equivalent to the recited value (e.g., having the same function or result). In some instances, the term “about” may include numerical values that are rounded to the nearest significant figure.

As used herein, “treat” and all its forms and tenses (including, for example, treat, treating, treated, and treatment) refer to both therapeutic treatment and prophylactic or preventative treatment. Those in need of treatment include those already with a pathological condition of the invention (e.g., microbial infection or immune related disease or disorder) as well as those in which a pathological condition of the invention is to be prevented.

As used herein, an “agent” is a molecular entity including, for example, a small molecule, nucleic acid (such as, siRNA, shRNA expression cassette, antisense DNA, antisense RNA), protein, peptide, antibody, antisense drug, or other biomolecule that is naturally made, synthetically made, or semi-synthetically made and is used alone or in combination with other therapies that can alleviate, reduce, ameliorate, prevent, or maintain in a state of remission clinical symptoms or diagnostic markers associated with a pathological condition of the invention (e.g., microbial infection or immune related disease or disorder).

As used herein, “infection” and all its forms and tenses (including, for example, infect and infected) is the presence or establishment of a microorganism in a host after the host has been exposed to the microorganism. A microorganism includes, for example, a bacterium, virus, biological warfare agent, fungi (e.g., molds, mildews, smuts, mushrooms, and yeasts), and protozoa (e.g., parasites). Infection further encompasses not only the initial infection, but also any subsequent infection, condition, or disease associated with the presence or establishment of the microorganism in the host.

II. The Present Invention

The invention is drawn to RNase-L-mediated antimicrobial activity. The invention uses microbial infection models such as B. anthracis and E. coli. As will be readily apparent to one of ordinary skill in the art, the inventors have discovered a novel role of RNase-L and methods of exploiting the role of RNase-L in innate immunity for the treatment of a microbial infection. A microbial infection includes infection caused by the exposure to an array of microorganisms including, for example, a bacterium, virus, biological warfare agent, fungi (e.g., molds, mildews, smuts, mushrooms, and yeasts), and protozoa (e.g., parasites).

Bacterial Infection

In certain embodiments the invention is draw to treating a bacterial infection. A bacterial infection can be caused by a myriad of bacteria and is marked by increases in bacterial load in the body. A bacterial infection can be caused by, for example, exposure to a bacterium and any species or derivative associated therewith, from, for example, any one or more of the following bacterium genera: Abiotrophia, Acaricomes, Acetitomaculum, Acetivibrio, Acetobacter, Acetobacterium, Acetobacteroides, Acetogenium, Acetohalobium, Acetomicrobium, Acetomonas, Acetonema, Achromobacter, Acidaminobacter, Acidaminococcus, Acidimicrobium, Acidiphilium, Acidithiobacillus, Acidobacterium, Acidocaldus, Acidocella, Acidomonas, Acidovorax, Acinetobacter, Acrocarpospora, Actinacidiphilus, Actinoacidiphilus, Actinoalloteichus, Actinobacillus, Actinobaculum, Actinobifida, Actinobispora, Actinocatenispora, Actinocorallia, Actinokineospora, Actinomadura, Actinomyces, Actinoplanes, Actinopolyspora, Actinopycnidium, Actinosporangium, Actinosynnema, Actinotelluria, Adhaeribacter, Aequorivita, Aerobacter, Aerococcus, Aeromicrobium, Aeromonas, Aestuariibacter, Afipia, Agarbacterium, Agitococcus, Agreia, Agrobacterium, Agrococcus, Agromonas, Agromyces, Ahrensia, Albidovulum, Alcaligenes, Alcanivorax, Algibacter, Algoriphagus, Alicycliphilus, Alicyclobacillus, Alishewanella, Alistipes, Alkalibacillus, Alkalibacter, Alkalibacterium, Alkalilimnicola, Alkalispirillum, Alkanindiges, Allisonella, Allobaculum, Allochromatium, Allofustis, Alteromonas, Alysiella, Aminobacter, Aminobacterium, Aminomonas, Ammonifex, Ammoniphilus, Amoebobacter, Amorphosporangium, Amphibacillus, Ampullariella, Amycolata, Amycolatopsis, Anaeroarcus, Anaerobacter, Anaerobaculum, Anaerobiospirillum, Anaerobranca, Anaerocellum, Anaerococcus, Anaerofilum, Anaerofustis, Anaerolinea, Anaeromusa, Anaerophaga, Anaeroplasma, Anaerosinus, Anaerostipes, Anaerotruncus, Anaerovibrio, Anaerovorax, Ancalomicrobium, Ancylobacter, Aneurinibacillus, Angiococcus, Angulomicrobium, Anoxybacillus, Antarctobacter, Aquabacter, Aquabacterium, Aquamicrobium, Aquaspirillum, Aquicella, Aquifex, Aquiflexum, Aquimonas, Arachnia, Arcanobacterium, Archangium, Arcicella, Arcobacter, Arenibacter, Arhodomonas, Arizona, Arsenicicoccus, Arsenophonus, Arthrobacter, Asanoa, Asiosporangium, Asticcacaulis, Atopobium, Atopococcus, Atopostipes, Aurantimonas, Aureobacterium, Avibacterium, Axonoporis, Azoarcus, Azohydromonas, Azomonas, Azomonotrichon, Azorhizobium, Azorhizophilus, Azospira, Azospirillum, Azotobacter, Bacillus, Bacterionema, Bacteriovorax, Bacterium, Bacteroides, Balnearium, Balneatrix, Bartonella, Bdellovibrio, Beggiatoa, Beijerinckia, Belliella, Belnapia, Beneckea, Bergeriella, Betabacterium, Beutenbergia, Bifidobacterium, Bilophila, Blastobacter, Blastochloris, Blastococcus, Blastomonas, Blastopirellula, Bogoriella, Bordetella, Borrelia, Bosea, Brachybacterium, Brachymonas, Brachyspira, Brackiella, Bradyrhizobium, Branhamella, Brenneria, Brevibacillus, Brevibacterium, Brevigemma, Brevundimonas, Brochothrix, Brucella, Bryantella, Budvicia, Bulleidia, Burkholderia, Buttiauxella, Butyribacterium, Butyrivibrio, Byssovorax, Caenibacterium, Caldanaerobacter, Calderobacterium, Caldicellulosiruptor, Caldilinea, Caldithrix, Caldocellum, Caloramator, Caloranaerobacter, Caminibacillus, Caminibacter, Caminicella, Campylobacter, Capnocytophaga, Carbophilus, Carboxydibrachium, Carboxydocella, Carboxydothermus, Cardiobacterium, Carnobacterium, Caryophanon, Caseobacter, Castellaniella, Catellatospora, Catellibacterium, Catenibacterium, Catenococcus, Catenuloplanes, Catenulospora, Caulobacter, Cedecea, Cellulomonas, Cellulophaga, Cellulosimicrobium, Cellvibrio, Centipeda, Cerasibacillus, Chainia, Chelatobacter, Chelatococcus, Chitinibacter, Chitinophaga, Chlorobaculum, Chlorobium, Chloroflexus, Chondrococcus, Chondromyces, Chromatium, Chromobacterium, Chromohalobacter, Chryseobacterium, Chryseomonas, Chrysiogenes, Citreicella, Citricoccus, Citrobacter, Clavibacter, Clavisporangium, Clostridium, Cobetia, Cohnella, Collimonas, Collinsella, Colwellia, Comamonas, Conchiformibius, Conexibacter, Coprothermobacter, Corallococcus, Coriobacterium, Corynebacterium, Couchioplanes, Crossiella, Cryobacterium, Cryptanaerobacter, Cryptobacterium, Cryptosporangium, Cupriavidus, Curtobacterium, Curvibacter, Cyclobacterium, Cystobacter, Cytophaga, Dactylosporangium, Dechloromonas, Dechlorosoma, Deferribacter, Defluvibacter, Dehalobacter, Dehalospirillum, Deinobacter, Deinococcus, Deleya, Delftia, Demetria, Dendrosporobacter, Denitrovibrio, Dermabacter, Dermacoccus, Dermatophilus, Derxia, Desemzia, Desulfacinum, Desulfarculus, Desulfatibacillum, Desulfitobacterium, Desulfoarculus, Desulfobacca, Desulfobacter, Desulfobacterium, Desulfobacula, Desulfobulbus, Desulfocapsa, Desulfocella, Desulfococcus, Desulfofaba, Desulfofrigus, Desulfofustis, Desulfohalobium, Desulfomicrobium, Desulfomonas, Desulfomonile, Desulfomusa, Desulfonatronovibrio, Desulfonatronum, Desulfonauticus, Desulfonema, Desulfonispora, Desulforegula, Desulforhabdus, Desulforhopalus, Desulfosarcina, Desulfospira, Desulfosporosinus, Desulfotalea, Desulfothermus, Desulfotignum, Desulfotomaculum, Desulfovibrio, Desulfovirga, Desulfurella, Desulfurobacterium, Desulfuromonas, Desulfuromusa, Dethiosulfovibrio, Devosia, Dialister, Diaphorobacter, Dichelobacter, Dichotomicrobium, Dickeya, Dictyoglomus, Dietzia, Diplococcus, Dokdoa, Dokdonella, Dokdonia, Dolosicoccus, Donghaeana, Dorea, Duganella, Dyadobacter, Dyella, Eberthella, Ectothiorhodospira, Edwardsiella, Eggerthella, Eikenella, Elizabethkingia, Elytrosporangium, Empedobacter, Enhygromyxa, Ensifer, Enterobacter, Enterococcus, Enterovibrio, Epilithonimonas, Eremococcus, Erwinia, Erysipelothrix, Erythrobacter, Erythromicrobium, Erythromonas, Escherichia, Eubacterium, Ewingella, Excellospora, Exiguobacterium, Faecalibacterium, Faenia, Falcivibrio, Ferrimonas, Ferrobacillus, Fervidobacterium, Filibacter, Filifactor, Filobacillus, Filomicrobium, Finegoldia, Flammeovirga, Flavimonas, Flavobacterium, Flectobacillus, Flexibacter, Flexistipes, Flexithrix, Fluoribacter, Fluviicola, Formivibrio, Francisella, Frankia, Frateuria, Friedmanniella, Frigoribacterium, Fulvimarina, Fulvimonas, Fundibacter, Fusibacter, Fusobacterium, Gaetbulibacter, Gaetbulimicrobium, Gaffkya, Gallibacterium, Gallicola, Garciella, Gardnerella, Gariaella, Gelidibacter, Gelria, Gemella, Gemmata, Gemmatimonas, Gemmobacter, Geobacillus, Geobacter, Geodermatophilus, Geopsychrobacter, Georgenia, Geospirillum, Geothermobacter, Geothrix, Geovibrio, Giesbergeria, Gillisia, Glaciecola, Globicatella, Gluconacetobacter, Gluconoacetobacter, Gluconobacter, Glycomyces, Goodfellowia, Gordona, Gordonia, Gracilibacillus, Granulicatella, Granulobacter, Grimontia, Guggenheimella, Gulosibacter, Haemophilus, Hafnia, Hahella, Halanaerobacter, Halanaerobium, Haliangium, Haliscomenobacter, Haloanaerobacter, Haloanaerobium, Halobacillus, Halobacteroides, Halocella, Halochromatium, Halococcus, Haloincola, Halolactibacillus, Halomonas, Halonatronum, Halorhodospira, Halothermothrix, Halothiobacillus, Halovibrio, Helcococcus, Helicobacter, Heliobacillus, Heliobacterium, Heliophilum, Heliorestis, Herbaspirillum, Herbidospora, Herpetosiphon, Hespellia, Hippea, Hirschia, Hoeflea, Holdemania, Holophaga, Hongiella, Hordeomyces, Hyalangium, Hydrocarboniphaga, Hydrogenivirga, Hydrogenobacter, Hydrogenobaculum, Hydrogenomonas, Hydrogenophaga, Hydrogenophilus, Hydrogenothermophilus, Hydrogenothermus, Hydrogenovibrio, Hylemonella, Hymenobacter, Hyphomicrobium, Hyphomonas, Idiomarina, Ignavigranum, Ilyobacter, Inflabilis, Inquilinus, Intrasporangium, Iodobacter, Isobaculum, Isochromatium, Isoptericola, Jahnia, Janibacter, Jannaschia, Janthinobacterium, Jensenia, Jeotgalicoccus, Jiangella, Jonesia, Kangiella, Kerstersia, Kibdellosporangium, Kibdelosporangium, Kineococcus, Kineosphaera, Kineosporia, Kingella, Kitasatoa, Kitasatospora, Kitasatosporia, Klebsiella, Kluyvera, Knoellia, Kocuria, Kofleria, Koserella, Kozakia, Kribbella, Kurthia, Kutzneria, Kytococcus, Labrys, Laceyella, Lachnobacterium, Lachnospira, Lactobacillus, Lactobacterium, Lactococcus, Lactosphaera, Lamprocystis, Lampropedia, Laribacter, Lautropia, Leadbetterella, Lebetimonas, Lechevalieria, Leclercia, Leeuwenhoekiella, Legionella, Leifsonia, Leisingera, Leminorella, Lentibacillus, Lentzea, Leptospirillum, Leptothrix, Leptotrichia, Leucobacter, Leuconostoc, Leucothrix, Levilinea, Levinea, Limnobacter, List, Listeria, Listonella, Loktanella, Lonepinella, Longispora, Lophomonas, Lucibacterium, Luteibacter, Luteimonas, Luteococcus, Lysobacter, Macrococcus, Macromonas, Magnetospirillum, Mahella, Malikia, Malonomonas, Mannheimia, Maribacter, Maricaulis, Marichromatium, Marinibacillus, Marinilabilia, Marinilactibacillus, Marinithermus, Marinitoga, Marinobacter, Marinobacterium, Marinococcus, Marinomonas, Marinospirillum, Marinovum, Marmoricola, Massilia, Megamonas, Megasphaera, Meiothermus, Melittangium, Mesonia, Mesophilobacter, Mesorhizobium, Methanomonas, Methylobacillus, Methylobacterium, Methylocapsa, Methylocella, Methylomicrobium, Methylomonas, Methylophaga, Methylophilus, Methylopila, Methylosarcina, Methylotenena, Methylovorus, Microbacterium, Microbispora, Microbulbifer, Micrococcus, Microcyclus, Microechinospora, Microellobosporia, Microlunatus, Micromonas, Micromonospora, Micropolyspora, Micropruina, Microscilla, Microsphaera, Microstreptospora, Microtetraspora, Microvirgula, Millisia, Mima, Mitsuokella, Mobiluncus, Modestobacter, Moellerella, Mogibacterium, Moorella, Moraxella, Moraxella, (Branhamella), Moraxella, (Moraxella), Morganella, Moritella, Muricauda, Muricoccus, Myceligenerans, Mycetocola, Mycobacterium, Mycoplana, Myroides, Myxococcus, Nakamurella, Nannocystis, Natroniella, Natronincola, Nautilia, Naxibacter, Neisseria, Nereida, Nesterenkonia, Nevskia, Nicoletella, Nitratifractor, Nitratireductor, Nitratiruptor, Nitrobacter, Nocardia, Nocardioides, Nocardiopsis, Nonomuraea, Novosphingobium, Obesumbacterium, Oceanibulbus, Oceanicaulis, Oceanicola, Oceanimonas, Oceanithermus, Oceanobacillus, Oceanobacter, Oceanomonas, Oceanospirillum, Ochrobactrum, Octadecabacter, Odontomyces, Oenococcus, Oerskovia, Oleiphilus, Oleispira, Oligella, Oligotropha, Olsenella, Opitutus, Orenia, Oribacterium, Ornithinicoccus, Ornithinimicrobium, Ornithobacterium, Ottowia, Oxalicibacterium, Oxalobacter, Oxalophagus, Oxobacter, Paenibacillus, Paludibacter, Pandoraea, Pannonibacter, Pantoea, Papillibacter, Paracoccus, Paracolobactrum, Paralactobacillus, Paraliobacillus, Parascardovia, Parasporobacterium, Parvibaculum, Parvopolyspora, Pasteurella, Pasteuria, Patulibacter, Paucibacter, Paucimonas, Pectinatus, Pectobacterium, Pediococcus, Pedobacter, Pelczaria, Pelobacter, Pelodictyon, Pelomonas, Pelospora, Pelotomaculum, Peptococcus, Peptoniphilus, Peptostreptococcus, Peredibacter, Persephonella, Persicivirga, Persicobacter, Petrimonas, Petrobacter, Petrotoga, Phaeobacter, Phaeospirillum, Phascolarctobacterium, Phenylobacterium, Phocoenobacter, Photobacterium, Photorhabdus, Phyllobacterium, Phytomonas, Pigmentiphaga, Pilimelia, Pimelobacter, Pirella, Pirellula, Planctomyces, Planifilum, Planobispora, Planococcus, Planomicrobium, Planomonospora, Planopolyspora, Planotetraspora, Plantibacter, Pleomorphomonas, Plesiocystis, Plesiomonas, Podangium, Polaribacter, Polaromonas, Polyangium, Polymorphospora, Pontibacillus, Porphyrobacter, Porphyromonas, Pragia, Prauserella, Prevotella, Proactinomyces, Promicromonospora, Promyxobacterium, Propionibacter, Propionibacterium, Propionicimonas, Propioniferax, Propionigenium, Propionimicrobium, Propionispira, Propionispora, Propionivibrio, Prosthecobacter, Prosthecochloris, Prosthecomicrobium, Protaminobacter, Proteiniphilum, Proteus, Protomonas, Providencia, Pseudaminobacter, Pseudoalteromonas, Pseudoamycolata, Pseudobutyrivibrio, Pseudoclavibacter, Pseudomonas, Pseudonocardia, Pseudoramibacter, Pseudorhodobacter, Pseudospirillum, Pseudoxanthomonas, Psychrobacter, Psychroflexus, Psychromonas, Psychroserpens, Pusillimonas, Pyxicoccus, Quadrisphaera, Rahnella, Ralstonia, Ramibacterium, Ramlibacter, Raoultella, Rarobacter, Rathayibacter, Reinekea, Renibacterium, Renobacter, Rhabdochromatium, Rheinheimera, Rhizobacter, Rhizobium, Rhizomonas, Rhodobacter, Rhodobium, Rhodoblastus, Rhodocista, Rhodococcus, Rhodocyclus, Rhodoferax, Rhodomicrobium, Rhodopila, Rhodoplanes, Rhodopseudomonas, Rhodospirillum, Rhodothalassium, Rhodothermus, Rhodovibrio, Rhodovulum, Riemerella, Rikenella, Robiginitalea, Roseateles, Roseburia, Roseiflexus, Roseinatronobacter, Roseobacter, Roseococcus, Roseospira, Roseospirillum, Roseovarius, Rothia, Rubritepida, Rubrivivax, Rubrobacter, Ruegeria, Ruminobacter, Ruminococcus, Saccharibacter, Saccharococcus, Saccharomonospora, Saccharophagus, Saccharopolyspora, Saccharothrix, Sagittula, Salana, Salegentibacter, Salibacillus, Salinibacter, Salinibacterium, Salinicoccus, Salinimonas, Salinispora, Salinivibrio, Salinospora, Salipiger, Salmonella, Samsonia, Sanguibacter, Saprospira, Sarcina, Sarraceniospora, Scardovia, Schineria, Schlegelella, Schwartzia, Sebekia, Sedimentibacter, Segniliparus, Seinonella, Sejongia, Selenomonas, Seliberia, Serinicoccus, Serpulina, Serratia, Shewanella, Shigella, Shinella, Shuttleworthia, Silanimonas, Silicibacter, Simonsiella, Simplicispira, Simsoniella, Sinorhizobium, Skermania, Slackia, Smaragdicoccus, Smithella, Sodalis, Soehngenia, Sorangium, Sphaerobacter, Sphaerophorus, Sphaerosporangium, Sphaerotilus, Sphingobacterium, Sphingobium, Sphingomonas, Sphingopyxis, Spirilliplanes, Spirillospora, Spirillum, Spirochaeta, Spirosoma, Sporacetigenium, Sporanaerobacter, Sporichthya, Sporobacter, Sporobacterium, Sporocytophaga, Sporohalobacter, Sporolactobacillus, Sporomusa, Sporosarcina, Sporotomaculum, Stackebrandtia, Staleya, Stanierella, Staphylococcus, Stappia, Starkeya, Stella, Stenotrophomonas, Sterolibacterium, Stigmatella, Stomatococcus, Streptacidiphilus, Streptimonospora, Streptoallomorpha, Streptoalloteichus, Streptobacillus, Streptobacterium, Streptococcus, Streptomonospora, Streptomyces, Streptomycoides, Streptosporangium, Streptoverticillium, Subdoligranulum, Subtercola, Succiniclasticum, Succinimonas, Succinispira, Succinivibrio, Sulfitobacter, Sulfobacillus, Sulfuricurvum, Sulfurihydrogenibium, Sulfurimonas, Sulfurospirillum, Sutterella, Suttonella, Syntrophobacter, Syntrophobotulus, Syntrophococcus, Syntrophomonas, Syntrophospora, Syntrophothermus, Syntrophus, Tatlockia, Tatumella, Taxeobacter, Taylorella, Teichococcus, Telluria, Tenacibaculum, Tepidibacter, Tepidimicrobium, Tepidimonas, Tepidiphilus, Terasakiella, Terrabacter, Terracoccus, Terrimonas, Tessaracoccus, Tetragenococcus, Tetrasphaera, Tetrathiobacter, Thalassobacillus, Thalassobacter, Thalassobius, Thalassolituus, Thalassomonas, Thauera, Thaxtera, Thermacetogenium, Thermaerobacter, Thermanaeromonas, Thermanaerovibrio, Thermicanus, Thermincola, Thermithiobacillus, Thermoactinomyces, Thermoanaerobacter, Thermoanaerobacterium, Thermoanaerobium, Thermoanaerolinea, Thermobacterium, Thermobacteroides, Thermobifida, Thermobispora, Thermobrachium, Thermochromatium, Thermocrinis, Thermocrispum, Thermodesulfatator, Thermodesulfobacterium, Thermodesulfobium, Thermodesulforhabdus, Thermodesulfovibrio, Thermoflavimicrobium, Thermohydrogenium, Thermomicrobium, Thermomonas, Thermomonospora, Thermonema, Thermonospora, Thermopolyspora, Thermosediminibacter, Thermosiculum, Thermosinus, Thermosipho, Thermosyntropha, Thermoterrabacterium, Thermotoga, Thermovenabulum, Thermovibrio, Thermus, Thetysia, Thialkalimicrobium, Thialkalivibrio, Thioalkalimicrobium, Thioalkalivibrio, Thiobaca, Thiobacillus, Thiobacter, Thiocapsa, Thiococcus, Thiocystis, Thiodictyon, Thiohalocapsa, Thiolamprovum, Thiomicrospira, Thiomonas, Thiopedia, Thioreductor, Thiorhodoccocus, Thiorhodococcus, Thiorhodovibrio, Thiosphaera, Thiothrix, Tindallia, Tissierella, Tolumonas, Trabulsiella, Treponema, Trichococcus, Trichotomospora, Truepera, Tsukamurella, Turicella, Turicibacter, unclassified, Ureibacillus, Uruburuella, Vagococcus, Varibaculum, Variovorax, Veillonella, Verrucomicrobium, Verrucosispora, Vibrio, Victivallis, Virgibacillus, Virgisporangium, Vitreoscilla, Vogesella, Volcaniella, Volucribacter, Vulcanibacillus, Vulcanithermus, Waksmania, Wautersia, Weeksella, Weissella, Williamsia, Wolinella, Woodsholea, Xanthobacter, Xanthomonas, Xenophilus, Xenorhabdus, Xylanibacterium, Xylanimicrobium, Xylanimonas, Xylella, Xylophilus, Yania, Yersinia, Yokenella, Zavarzinia, Zimmermannella, Zobellia, Zoogloea, Zooshikella, Zymobacter, Zymobacterium, Zymomonas, and Zymophilus.

In certain embodiments of the invention methods of treating a bacterial infection comprise administering to a subject in need thereof one or more agents of the invention (e.g., an agent that increases the activity RNase-L or an agent that increases the expression of RNase-L). In other embodiments, administering one or more agents of the invention can also be administered with, for example, a bacterial therapy consisting of or comprising the administration of, for example, Penicillin G Pot in Dextrose IV, Penicillin G Potassium in D5W IV, Penicillin G Potassium Inj, Penicillin G Sodium Inj, Pfizerpen-G Inj, ADOXA Oral, ADOXA Pak Oral, Cleeravue-M Convenience Kit Misc, Declomycin Oral, Demeclocycline Oral, Doryx Oral, Doxycycline Calcium Oral, Doxycycline Hyclate IV, Doxycycline Hyclate Oral, Doxycycline Monohydrate Oral, Doxy-Lemmon Oral, Dynacin Oral, Minocin Oral, Minocin Prof Acne Care Misc, Minocycline Oral, Minocycline-Eyelid Cleanser #1 Misc, Minocycline-Wipes,Emolnt,&Mask Misc, Monodox Oral, Myrac Oral, Oxytetracycline IM, Sumycin 250 Oral, Sumycin 500 Oral, Sumycin Oral, Terramycin IM, Terramycin IM IM, Tetracycline Oral, Vibramycin Oral, Vibra-Tabs Oral, Cleocin in D5W IV, Cleocin Inj, Cleocin IV, Cleocin Oral, Clindamycin HCl Oral, Clindamycin Palmitate Oral, Clindamycin Phosphate Inj, Clindamycin Phosphate IV, E.E.S. 200 Oral, E.E.S. 400 Oral, E.E.S. Granules Oral, E-Mycin Oral, Eryc Oral, EryPed 200 Oral, EryPed 400 Oral, EryPed Oral, Ery-Tab Oral, Erythrocin IV, Erythrocin Stearate Oral, Erythromycin Ethylsuccinate Oral, Erythromycin Lactobionate IV, Erythromycin Oral, Erythromycin Stearate Oral, PCE Oral, Penicillin V Potassium Oral, Dapsone Oral, Biaxin Oral, Cefpodoxime Oral, Ceftin Oral, Cefuroxime Axetil Oral, Cipro I.V. IV, Cipro in D5W IV, Ciprofloxacin in D5W IV, Ciprofloxacin IV, Clarithromycin Oral, Levaquin in D5W IV, Levaquin IV, Levaquin Leva-Pak Oral, Levaquin Oral, Levofloxacin in D5W IV, Levofloxacin IV, Levofloxacin Oral, Vantin Oral, Cefixime Oral, Suprax Oral, Amoclan Oral, Amoxicillin Oral, Amoxicillin-Pot Clavulanate Oral, Amoxil Oral, Ampicillin Oral, Augmentin Oral, Augmentin XR Oral, Avelox ABC Pack Oral, Avelox in NaCl (Iso-osmotic) IV, Avelox Oral, Azithromycin Oral, Cefprozil Oral, Cefzil Oral, Moxifloxacin in Saline IV, Moxifloxacin Oral, Trimox Oral, Zithromax Oral, Zithromax TRI-PAK Oral, Zithromax Z-Pak Oral, Zmax Oral, Biaxin XL Oral, Biaxin XL Pak Oral, Cefdinir Oral, Cipro Oral, Ciprofloxacin Oral, Clarithromycin ER Oral, Omnicef Oral, Flagyl Oral, Metro I.V. IV, Metronidazole in NaCl (Iso-os) IV, Metronidazole Oral, Metryl Oral, Ticar in D5W IV, Ticar In Dextrose IV, Ticar Inj, Ticar IV, Ticarcillin in D5W IV, Ticarcillin Inj, Ticarcillin IV, AK-Tob Opht, AK-Trol Opht, Blephamide Opht, Blephamide S.O.P. Opht, Cortisporin Opht, Dexacidin Opht, Dexasporin Opht, Garamycin Opht, Genoptic Opht, Genoptic S.O.P. Opht, Gentak Opht, Gentamicin Opht, Gentamicin-Prednisolone Opht, Gentasol Opht, Maxitrol Opht, Methadex Opht, Neomycin-Bacitracin-Poly-HC Opht, Neomycin-Polymyxin-Dexameth Opht, Neomycin-Polymyxin-Prednisolon Opht, Poly-Dex Opht, Poly-Pred Opht, Pred-G Opht, Pred-G S.O.P. Opht, Sulfacetamide-Prednisolone Opht, TobraDex Opht, Tobramycin Sulfate Opht, Tobramycin-Dexamethasone Opht, Tobrasol Opht, Tobrex Opht, Triple Antibiotic-HC Opht, Vasocidin Opht, AK-Poly-Bac Opht, AK-Spore Opht, Bacitracin Opht, Bacitracin-Polymyxin B Opht, Bleph-10 Opht, Erythromycin Opht, Neocidin Opht, Neomycin-Bacitracin-Polymyxin Opht, Neomycin-Polymyxin-Gramicidin Opht, Neosporin Opht, Ocutricin Opht, Polycin B Opht, Polysporin Opht, Romycin Opht, Sulfac Opht, Sulfacetamide Sodium Opht, Triple Antibiotic Opht, Ciloxan Opht, Ciprofloxacin Opht, Gatifloxacin Opht, Levofloxacin Opht, Moxifloxacin Opht, Ocuflox Opht, Ofloxacin Opht, Oxytetracycline-Polymyxin B Opht, Polymyxin B Sul-Trimethoprim Opht, Polytrim Opht, Quixin Opht, Terramycin Opht, Trimethoprim-Polymyxin B Opht, Vigamox Opht, Zymar Opht, Ampicillin Sodium Inj, Ampicillin Sodium IV, Ancef Inj, Cefazolin in D5W IV, Cefazolin in Dextrose (Iso-os) IV, Cefazolin in Normal Saline IV, Cefazolin Inj, Cefazolin IV, Imipenem-Cilastatin IV, Penicillin G Procaine IM, Primaxin IV IV, Totacillin-N Inj, Totacillin-N IV, Vancocin in Dextrose IV, Vancomycin in Dextrose IV, Vancomycin in Normal Saline IV, Vancomycin IV, Bactrim DS Oral, Bactrim Oral, Septra DS Oral, Septra Oral, SMZ-TMP DS Oral, Sulfatrim Oral, Trimethoprim-Sulfamethoxazole IV, Trimethoprim-Sulfamethoxazole Oral, Azactam Inj, Azactam-Iso-osmotic Dextrose IV, Aztreonam in Dextrose(IsoOsm) IV, Aztreonam Inj, Cephalexin Oral, Keflex Oral, Amikacin Inj, Amikin Inj, Cefotaxime in D5W IV, Cefotaxime Inj, Cefotaxime IV, Ceftazidime Inj, Ceftazidime-Dextrose (Iso-osm) IV, Ceftriaxone Inj, Ceftriaxone IV, Ceftriaxone-Dextrose (Iso-osm) IV, Cefuroxime in Sterile Water IV, Cefuroxime Sodium in D5W IV, Cefuroxime Sodium Inj, Cefuroxime-Dextrose (Iso-osm) IV, Claforan in D5W IV, Claforan Inj, Claforan IV, Fortaz in D5W IV, Fortaz Inj, Fortaz IV, Garamycin Inj, Gentamicin (Pediatric) Inj, Gentamicin in Normal Saline IV, Gentamicin in Saline (Iso-osm) IV, Gentamicin Inj, Gentamicin Sulfate (PF) IV, Meropenem IV, Nebcin In Dextrose IV, Rocephin Inj, Rocephin IV, TAZICEF Inj, TAZICEF IV, Tobramycin in D5W IV, Tobramycin in NS IV, Tobramycin Sulfate Inj, Tobramycin Sulfate IV, Zinacef in D5W IV, Zinacef in Dextrose (Iso-osm) IV, Zinacef in Sterile Water IV, Zinacef Inj, Zinacef IV, Azithromycin hydrogen citrate IV, Azithromycin IV, Cefditoren Pivoxil Oral, Cefepime Inj, Cefepime IV, Cefoxitin in 2.2% Dextrose IV, Cefoxitin in 3.9% Dextrose IV, Cefoxitin in Dextrose, Iso-osm IV, Cefoxitin Inj, Cefoxitin IV, Ertapenem Inj, Factive Oral, Gemifloxacin Oral, Imipenem-Cilastatin IM, Invanz Inj, Kanamycin Inj, Maxipime Inj, Maxipime IV, Mefoxin in Dextrose (Iso-osm) IV, Mefoxin IV, Piperacillin-Tazobactam IV, Piperacillin-Tazobactam-Dextrs IV, Primaxin IM IM, Spectracef Oral, Ticarcillin-Clavulanate IV, Timentin IV, Zithromax IV, Zosyn in Dextrose (Iso-osm) IV, Zosyn IV, Cefizox in Dextrose (Iso-osm) IV, Ceftizoxime-Dextrose (Iso-osm) IV, Piperacillin in D5W IV, Piperacillin Inj, Piperacillin IV, Pipracil in D5W IV, Pipracil In Dextrose IV, Polymyxin B Sulfate Inj, Cipro XR Oral, Ciprofloxacin (HCl-Betaine) Oral, Streptomycin IM, Chloramphenicol Sod Succinate IV, Cedax Oral, Cefaclor Oral, Ceftibuten Oral, Floxin I.V. in D5W IV, Floxin Oral, Ofloxacin in D5W IV, Ofloxacin Oral, KETEK Oral, Ketek Pak Oral, Telithromycin Oral, CUBICIN IV, Daptomycin IV, Linezolid IV, Linezolid Oral, Zyvox IV, Zyvox Oral, Dicloxacillin Oral, Colistimethate Sodium Inj, Coly-Mycin M Inj, Augmentin ES-600 Oral, Cephradine Oral, Erythromycin-Sulfisoxazole Oral, Gantrisin Pediatric Oral, Pediazole Oral, Primsol Oral, Sulfisoxazole Acetyl Oral, Trimethoprim Oral, Velosef Oral, Ceclor Oral, Raniclor Oral, Carimune NF Nanofiltered IV, Flebogamma DIF IV, Flebogamma IV, Gammagard Liquid IV, Gammagard S/D IV, Gammagard S-D (IgA<1 ug/mL) IV, Immune Glob(IGG)(Hum)-Maltose IV, Immune Globulin (Human) (IGG) IV, Iveegam En IV, Octagam IV, Polygam S/D IV, Venoglobulin-S IV, Bactroban Nasal Nasl, Mupirocin Calcium Nasl, Bactroban Top, Centany Top, Mupirocin Top, Lincocin Inj, Lincoject Inj, Lincomycin Inj, Penicillin G Benzathine & Proc IM, Bicillin C-R IM, Cefadroxil Oral, Duricef Oral, Pneumococcal 7-Val Conj Vacc IM, Prevnar IM, Cipro HC Otic, Ciprofloxacin-Hydrocortisone Otic, Cipro HC Otic, CIPRODEX Otic, Ciprofloxacin-Dexamethasone Otic, Floxin Otic, Ofloxacin Otic, Nafcillin in D2.4W IV, Nafcillin in D5W IV, Nafcillin Inj, Nafcillin IV, Nallpen in D2.4W IV, Nallpen in D5W IV, Nallpen In Dextrose IV, Nallpen Inj, Nallpen IV, Oxacillin in Dextrose IV, Oxacillin Inj, Oxacillin IV, Rifadin IV, Rifadin Oral, Rifampin IV, Rifampin Oral, Rimactane Oral, Ampicillin-Sulbactam Inj, Ampicillin-Sulbactam IV, Unasyn Inj, or Unasyn IV, alone or in combination with one or more of the foregoing.

In other embodiments, the present invention relates to adminisering compounds as disclosed in PCT Published Application No. WO 2007/127212 and U.S. Patent Application Ser. No. 60/759,069, which is incorporated by reference in its entirety. The present invention is the first disclosure to link RNase L activity with cathespin E expression and function and the small molecules disclosed in PCT Published Application No. WO 2007/127212 and U.S. Provisional Application Ser. No. 60/759,069 could be use to treat bacterial infection.

In particular, the present invention comprises adminsitering compounds of Formula I for treatment of bacterial infection.

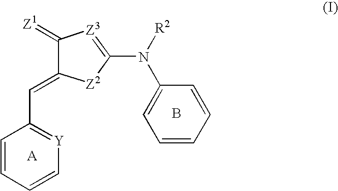

wherein,

Ring A and Ring B are optionally and independently substituted at any one or more substitutable ring carbon atoms;

Y is CH, N or N⁺—O⁻;

Z¹ and Z² are independently O or S;

Z³ is CR¹ or N;

R¹ is —H, —C(O)H, —C(O)R²⁰, —C(O)OR³⁰ or a C1-C5 alkyl group optionally substituted with one or more groups selected from halogen, hydroxyl, —OR²⁰, nitro, cyano, —C(O)H, —C(O)R²⁰, —C(O)OR³⁰, —OC(O)H and —OC(O)R²⁰ or R¹ is a group represented by the following structural formula:

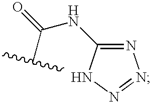

R² is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H or —OC(O)R²⁰;

each R²⁰ is independently C1-C3 alkyl or C1-C3 haloalkyl; and

R³⁰ is C1-C3 alkyl, C1-C3 haloalkyl or a group represented by a structural formula selected from:

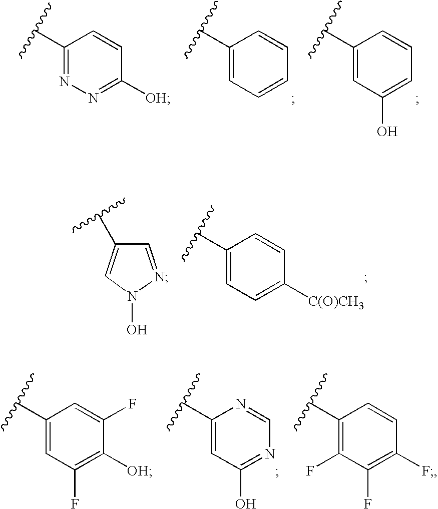

or a pharmaceutical salt thereof.

In specific embodiments of methods using compounds of Formula I, Z¹ is O and Z² is S.

In further specific embodiments, methods using compounds of Formula I include at least one compound selected from the group consisting of:

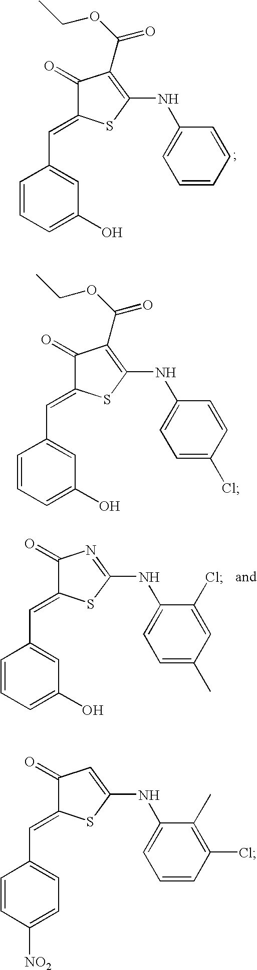

or a pharmaceutical salt thereof.

In even further specific embodiments, the methods of using compounds of Formula I include the compounds:

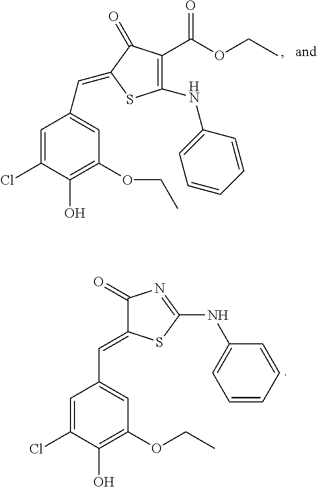

In other embodiments, the methods comprise adminisering compounds of Formula II to treat bacterial infections, or pharamceutical salts thereof.

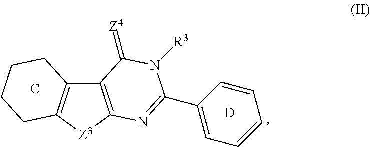

wherein,
Z³ and Z⁴ are independently O or S,
Ring C and Ring D are optionally and independently substituted at any one or more substitutable ring carbon atoms;
R³ is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰; and
each R²⁰ is independently C1-C3 alkyl or haloalkyl.

In specific embodiments, the methods of using compounds of Formula II include at least one compound selected from the group consisting of,

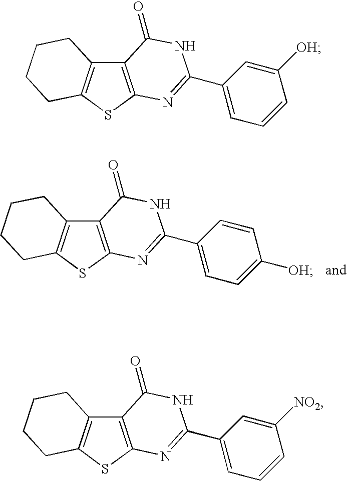

or a pharmaceutical salt thereof.

In other embodiments, the methods comprise adminisering compounds of Formula III to treat bacterial infections, or pharmaceutical salts thereof.

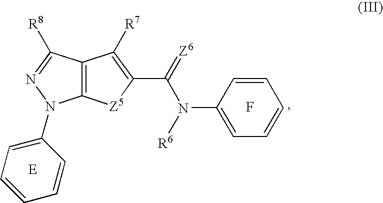

wherein,
Z⁵ and Z⁶ are independently O or S;
Ring E and Ring F are optionally and independently substituted at any one or more substitutable ring carbon atoms;
R⁶ is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R⁷ and R⁸ are independently —H, a C1-C5 alkyl group or a C1-C5 haloalkyl group; and
each R²⁰ is independently C1-C3 alkyl or haloalkyl.

In specific embodiments, the methods of using compounds of Formula III include the compound:

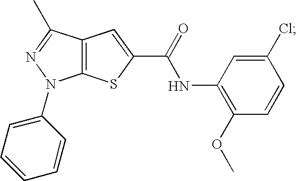

or a pharmaceutically acceptable salt thereof.

In other embodiments, the methods comprise adminisering compounds of Formula IV to treat bacterial infections, or pharmaceutical salts thereof.

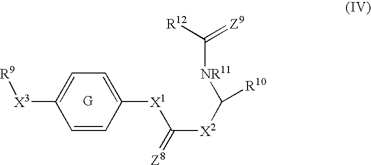

wherein,
X¹ and X² are independently CH₂, NH or O;
X³ is —O—C(O)—, —O—C(S)—, —S—C(O)—, —S—C(S)—, —C(O)—, C(S)—, —CH₂—, —CH(CH₃)—, —NHC(O)—, —C(O)NH—, —NHC(S)— or —C(S)NH—;
Z⁸ and Z⁹ are independently S or O;
Ring G is optionally substituted at any one or more substitutable ring carbon atoms;
R⁹ is a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R¹⁰ and R¹¹ are independently —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R¹² is —H; a C1-C5 alkyl group optionally substituted with one or more groups represented by R²¹; a monocyclic aromatic group optionally substituted at any one or more substitutable ring carbon atoms with a group represented by R²²; or a monocyclic C1-C3 aralkyl group optionally substituted at any one or more substitutable ring carbon atoms with R²³;
each R²⁰ is independently C1-C3 alkyl or C1-C3 haloalkyl;
each R²¹ is independently halogen, hydroxyl, —OR²⁰, nitro, cyano, —C(O)H, —C(O)R²⁰, —C(O)OR²⁰, —OC(O)H or —OC(O)R²⁰;
each R²² and R²³ is independently C1-C3 alkyl, C1-C3 haloalkyl, nitro, cyano, hydroxy, —OR²⁴, —C(O)H, —C(O)R²⁴, —C(O)OR²⁴, —OC(O)H, —OC(O)R²⁴ or C1-C3 alkyl substituted with hydroxyl, —OR²⁴, keto, —C(O)OR²⁴, —OC(O)H or —OC(O)R²⁴ and
R²⁴ is C1-C3 alkyl or C1-C3 haloalkyl,

In specific embodiments, the methods of using compounds of Formula IV include at least one compound selected from the group consisting of,

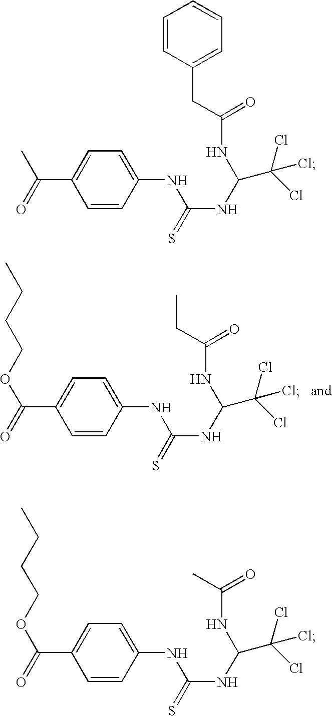

or a pharmaceutically acceptable salt thereof.

In specific embodiments of treating bacterial infections, the methods comprise treating a bacterial infection in a subject, wherein the subject does not have a concomitant viral infection. Alternatively, the subject is not exhibiting symptoms of a viral infection. A healthcare worker can easily assess symptoms of a viral infection. Of course, symptoms of viral infections vary from one virus to another, but common symtpoms include sore throat, runny nose, fatigue, headache, muscle aches, vomiting, abdominal discomfort, and diarrhea. A viral infection, or the lack thereof, can be confirmed with a variety of well-known techniques including but not limited to, blood tests to check for antibodies or antigens, cultures of blood, bodily fluid, or other material taken from the subject, spinal taps to examine the cerebrospinal fluid, genetic tests, such as a polymerase chain reaction (PCR) to accurately identify the virus, and magnetic resonance imaging (MRI) that can detect increased swelling in the temporal lobes. In a more specific embodiment, the subject is diagnosed with having only a bacterial infection and not a viral infection. Methods of assessing and diagnosing a bacterial infection are routine in the art and many of the same methods for determing the lack of a viral infection can also be used to determine and monitor bacterial infection. For example, assessing bacterial load or titer or other methods of assessing levels of bacterial infection can be performed both before and after administration of the agents of the present invention. Methods of assessing bacterial infection also include monitoring symptoms of bacterial infection in the subject either before and/or after administration of any of the agents of the present invention. Again, the symptoms of a bacterial infection vary from one type of bacteria to another and a healthcare worker can track these symptomps.

Viral Infection

In certain embodiments the invention is draw to treating a viral infection. A viral infection can be caused by a myriad of viruses and is marked by increases in viral load in the body. A viral infection can be caused by, for example, exposure to a virus (including, for example, a DNA or RNA virus) and any species or derivative associated therewith, from, for example, any one or more of the following virus families: Adenoviridae, Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae, Corticoviridae, Cystoviridae, Deltavirus, Dianthovirus, Enamovirus, Filoviridae, Flaviviridae, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteovirus, Machlomovirus, Marafivirus, Microviridae, Mononegavirales, Myoviridae, Necrovirus, Nodaviridae, Orthomyxoviridae, Papovaviridae, Paramyxoviridae, Partitiviridae, Parvoviridae, Phycodnaviridae, Picornaviridae, Plasmaviridae, Podoviridae, Polydnaviridae, Potexvirus, Potyviridae, Poxviridae, Prions, Reoviridae, Retroviridae, Rhabdoviridae, Rhizidiovirus, Satellites (for example, DNA satellite viruses and RNA satellite viruses), Sequiviridae, Siphoviridae, Sobemovirus, Tailed Phages, Tectiviridae, Tenuivirus, Tetraviridae, Tobamovirus, Tobravirus, Togaviridae, Tombusviridae, Totiviridae, Trichovirus, Tymovirus, Umbravirus, and Viroids.

In certain embodiments of the invention methods of treating viral infection comprise administering to a subject in need thereof one or more agents of the invention (e.g., an agent that increases the activity RNase-L or an agent that increases the expression of RNase-L). In other embodiments, administering one or more agents of the invention can also be administered with, for example, a viral therapy consisting of or comprising the administration of, for example, Interferon Alfa-2B Inj, Interferon Alfa-2B SubQ, Intron A Inj, Intron A SubQ, Foscarnet IV, Foscavir IV, Epoetin Alfa Inj, Epogen Inj, Procrit Inj, Megace ES Oral, Megace Oral Oral, Megestrol Oral, Adefovir Oral, Baraclude Oral, Entecavir Oral, Epivir HBV Oral, Hepsera Oral, Lamivudine Oral, Pegasys Convenience Pack SubQ, Pegasys SubQ, Peginterferon Alfa-2a SubQ, Telbivudine Oral, Tyzeka Oral, Interferon Alfa-2A SubQ, Roferon-A SubQ, Ribavirin Inhl, Virazole Inhl, Acyclovir Oral, Acyclovir Sodium IV, Zovirax Oral, Corticotropin Inj, Famciclovir Oral, Famvir Oral, Valacyclovir Oral, Valtrex Oral, Acthar H.P. Inj, Abacavir Oral, Abacavir-Lamivudine Oral, Abacavir-Lamivudine-Zidovudine Oral, Agenerase Oral, Amprenavir Oral, Aptivus Oral, Atazanavir Oral, ATRIPLA Oral, Combivir Oral, Crixivan Oral, Darunavir Oral, Delavirdine Oral, Didanosine Oral, Efavirenz Oral, Efavirenz-Emtricitabin-Tenofov Oral, Emtricitabine Oral, Emtricitabine-Tenofovir Oral, Emtriva Oral, Enfuvirtide SubQ, Epivir Oral, Epzicom Oral, Fosamprenavir Oral, Fuzeon SubQ, Hivid Oral, Indinavir Oral, Invirase Oral, Kaletra Oral, Lamivudine-Zidovudine Oral, LEXIVA Oral, Lopinavir-Ritonavir Oral, Nelfinavir Oral, Nevirapine Oral, Norvir Oral, Norvir Soft Gelatin Oral, Prezista Oral, Rescriptor Oral, Retrovir IV, Retrovir Oral, REYATAZ Oral, Ritonavir Oral, Saquinavir Mesylate Oral, Stavudine Oral, Sustiva Oral, Tenofovir Disoproxil Fumarate Oral, Tipranavir Oral, Trizivir Oral, Truvada Oral, Videx 2 gram Pediatric Oral, Videx 4 gram Pediatric Oral, Videx EC Oral, Viracept Oral, Viramune Oral, Viread Oral, Zalcitabine Oral, Zerit Oral, Ziagen Oral, Zidovudine IV, Zidovudine Oral, Seasonal flu shot, Amantadine Oral, Flumadine Oral, Rimantadine Oral, GARDASIL IM, Human Papillomavirus Vacc,Qval IM, Palivizumab IM, or Synagis IM, alone or in combination with any one or more of the foregoing.

Biological Warfare Agents

In certain embodiments the invention is draw to treating pathological conditions associated with an infectious or toxic biological warfare agent. A biological warfare agent includes, for example, Anthrax (Bacillus anthracis), Arenaviruses, Botulism (including, for example, Clostridium botulinum toxin types A through G), Brucella species (brucellosis), Burkholderia mallei (glanders), Burkholderia pseudomallei (melioidosis), Chlamydia psittaci (psittacosis), Cholera (Vibrio cholerae), Clostridium perfringens (Epsilon toxin), Coxiella burnetii (Q fever), Cryptosporidium parvum, Ebola virus hemorrhagic fever, E. coli O157:H7 (Escherichia coli), Emerging infectious diseases (including, for example, Nipah virus and hantavirus), Epsilon toxin of Clostridium perfringens, Filoviruses, Food safety threats (including, for example, Salmonella species, Escherichia coli O157:H7, and Shigella), Francisella tularensis (tularemia), Lassa fever, Marburg virus hemorrhagic fever, Plague (Yersinia pestis), Ricin toxin from Ricinus communis (castor beans), Rickettsia prowazekii (typhus fever), Salmonella species (salmonellosis), Salmonella Typhi (typhoid fever), Shigella (shigellosis), Smallpox (variola major), Staphylococcal enterotoxin B, Toxic syndrome, Viral encephalitis (including, for example, alphaviruses [including, for example, Venezuelan equine encephalitis, eastern equine encephalitis, western equine encephalitis]), Viral hemorrhagic fevers (filoviruses [including, for example, Ebola, and Marburg] and arenaviruses [including, for example, Lassa and Machupo]), and Water safety threats (including, for example, Vibrio cholerae, Cryptosporidium parvum), and Yersinia pestis (plague)).

Gene Delivery

In certain embodiments the invention is drawn to treating a microbial infection by administering an agent that increases the expression of RNase-L. In other embodiments, the agent that increases the expression of RNase-L comprises a vector comprising a polynucleotide encoding RNase-L or encoding a functional part thereof. The teachings as discussed herein and throughout are also germane to the invention wherein the invention is drawn to administering an agent that increases the activity of RNase-L wherein said agent comprises a nucleic acid or other means that is dependent on a nucleic acid.

As used herein, “vector” refers to a vehicle or other mechanism by which gene delivery can be accomplished. In certain embodiments, gene delivery can be achieved by a number of mechanisms including, for example, vectors derived from viral and non-viral sources, cation complexes, nanoparticles (including, for example, ormosil and other nano-engineered, organically modified silica, and carbon nanotubes; see for example, Panatarotto et al., Chemistry & Biology. 2003;10:961-966; Mah et al., Mol Therapy. 2000;1:S239; Salata et al., J Nanobiotechnology. 2004; 2:3) physical methods, or any combination thereof.

In certain embodiment, the invention is drawn to gene delivery comprising the use of viral vectors. Viruses are obligate intra-cellular parasites, designed through the course of evolution to infect cells, often with great specificity to a particular cell type. Viruses tend to be very efficient at transfecting their own DNA into the host cell, which is expressed to produce viral proteins. This characteristic and others, make viruses desirable and viable vectors for gene delivery. Viral vectors include both replication-competent and replication-defective vectors derived from various viruses. Viral vectors can be derived from a number of viruses, including, for example, polyoma virus, sindbis virus, fowlpox virus (UK 2,211,504 published 5 Jul. 1989), adenovirus and other viruses from the Adenoviridae family, adeno-associated virus and other viruses from the Parvoviridae family, herpes virus, vaccinia virus, alpha-virus, human immunodeficiency virus, papilloma virus, avian virus, cytomegalovirus, retrovirus, hepatitis-B virus, simian virus (including, for example, SV40), and chimeric viruses of any of the foregoing (including, for example, chimeric adenovirus). Though a number of viral vectors can accomplish gene delivery, interest has concentrated on a finite number of viral vectors, including, for example, those derived from retrovirus, adenovirus, adeno-associated virus, and herpes virus. Examples of viral vectors include, for example, AAV-MCS (adeno-associated virus), AAV-MCS2 (adeno-associated virus), Ad-Cla (E1/E3 deleted adenovirus), Ad-BGFP-Cla (E1/E3 deleted adenovirus), Ad-TRE (E1/E3 deleted adenovirus), MMP (MPSV/MLV derived retrovirus), MMP-iresGFP (MPSV/MLV derived retrovirus), MMP-iresGFPneo (MPSV/MLV derived retrovirus), SFG-TRE-ECT3 (3′ Enhancer deleted, MLV derived retrovirus), SFG-TRE-IRTECT3 (3′ Enhancer deleted, MLV derived retrovirus), HRST (3′ Enhancer deleted HIV derived retrovirus), simian adenovirus and chimeric adenovirus (see, for example, US Patent Publication Nos. 20060211115, 20050069866, 20040241181, 20040171807, 20040136963, and 20030207259).

In other embodiments, gene delivery also includes vectors comprising polynucleotide complexes comprising cyclodextrin-containing polycations (CDPs), other cationic non-lipid complexes (polyplexes), and cationic lipids complexes (lipoplexes) as carriers for gene delivery, which condense nucleic acids into complexes suitable for cellular uptake (see, for example, U.S. Pat. No. 6,080,728; Liu et al., Current Medicinal Chemistry, 2003, 10, 1307-1315; Gonazalez et al., Bioconjugate Chemistry 6:1068-1074 (1999); Hwang et al., Bioconjugate Chemistry 12:280-290 (2001)). A systems approach to prepare complexes and modify them with stabilizing and targeting components that result in stable, well-defined DNA- or RNA-containing complexes are suitable for in vivo administration. For example, polycations containing cyclodextrin can achieve high transfection efficiencies while remaining essentially non-toxic. A number of these complexes have been prepared that include variations in charge spacing, charge type, and sugar type (e.g., a spacing of six methylene units between adjacent amidine groups within the comonomer gave the best transfection properties). Other polyplexes comprise, for example, polyethyleneimime (available from Avanti Lipids), polylysine (available from Sigma), polyhistidine (Sigma), and SUPERFECT (available from Qiagen) (cationic polymer carriers for gene delivery in vitro and in vivo has been described in the literature, for example, by Goldman et al., Nature BioTechnology, 15:462 (1997)). Most polyplexes consist of cationic polymers and their complex production is regulated by ionic interactions. One large difference between the methods of action of polyplexes and lipoplexes is that some polyplexes cannot release their polynucleotides into the cytoplasm, which necessitates co-transfection with an endosome-lytic agent (to lyse the endosome that is made during endocytosis, the process by which a polyplex enters the cell) such as, for example, inactivated adenovirus. However this is not always the case, for example, polyplexes comprising polyethylenimine have their own method of endosome disruption as does chitosan and trimethylchitosan.

Lipoplexes (also known as cationic liposomes) function similar to polyplexes and are complexes comprising positively charged lipids. Lipoplexes are increasingly being used in gene therapy due to their favorable interactions with negatively charged DNA and cell membranes, as well as due to their low toxicity. Due to the positive charge of cationic lipids they naturally complex with the negatively charged DNA. Also as a result of their charge they interact with the cell membrane, endocytosis of a lipoplex occurs and the polynucleotide of interest is released into the cytoplasm. The cationic lipids also protect against degradation of the polynucleotide by the cell. The use of cationic lipids for gene delivery was initiated by Felgner and colleagues in 1987 who reported that liposomes consisting of N-[1-(2,3-dioleyloxy) propyl]-N,N,N-trimethylammonium chloride (DOTMA) and dioleoylphosphatidylethanolamine (DOPE) were capable of facilitating effective polynucleotide transfer across cell membranes, resulting in high level expression of the encoded gene (Felgner et al., PNAS (1987) 84: 7413-7417). Since this seminal work, many new cationic lipids have been synthesized and have been shown to possess similar transfection activity, many of which are summarized by Balaban et al. (Expert Opinion on Therapeutic Patents (2001), 11(11): 1729-1752).

In other embodiments, gene delivery of the invention also includes vectors encompassing physical approaches for gene transfer into cells in vitro and in vivo (Gao et al., AAPS Journal. 2007; 9(1): E92-E104). Physical approaches induce transient injuries or defects in cell membranes so that DNA can enter the cells by diffusion. Gene delivery by physical approaches include, for example, needle injection of naked DNA (see, for example, Wolff et al., Science. 1990; 247:1465-1468), electroporation (see, for example, Heller et al., Expert Opin Drug Deliv. 2005;2:255-268; Neumann et al., EMBO J. 1982;1:841-845), gene gun (see, for example, Yang et al., PNAS 1990;87:9568-9572; Yang et al., Nat Med. 1995;1:481-483), ultrasound (see, for example, Lawrie et al., Gene Ther. 2000;7:2023-2027), hydrodynamic delivery (see, for exmple, Liu et al., Gene Ther. 1999;6:1258-1266; Zhang et al., Hum Gene Ther. 1999;10:1735-1737), and laser-based energy (see, for example, Sagi et al., Prostate Cancer Prostatic Dis. 2003;6(2):127-30).

In other embodiments, gene delivery of the invention also includes bactofection (see, for example, Palffy et al., Gene Ther. January 2006; 13(2):101-5; Loessner et al., Expert Opin Biol Ther. February 2004; 4(2):157-68; Pilgrim et al., Gene Ther. November 2003; 10(24):2036-45; Weiss et al., Curr Opin Biotechnol. October 2001; 12(5):467-72; US Patent Application Publication No. 20030153527). Bacteria-mediated transfer of plasmid DNA into mammalian cells (i.e., bactofection) is a potent approach to express plasmid-encoded heterologous proteins (including, for example, therapeutic proteins, protein antigens, hormones, toxins, and enzymes) in a large set of different cell types in mammals. This mechanism of gene delivery uses bacteria for the direct transfer of nucleic acids into a target cell or cells. Transformed bacterial strains deliver the genes localized on plasmids into the cells, where these genes are then expressed. Generally, the method of bactofection comprises using transformed invasive bacteria as a vector to transport genetic material, which is in the form of, for example, a plasmid comprising sequences needed for the transcription and translation of the protein of interest. For example, bactofection comprises the steps of: (a) transforming invasive bacteria to contain plasmids carrying the transgene; (b) the transformed bacteria penetrates into the cells; (c) vectors are destructed or undergo lysis, which is induced by the presence of the bacteria in the cytoplasm, and release plasmids carried; and (d) the released plasmids get into the nucleus whereupon the transgene is expressed. Bacteria used in bactofection is preferably non-pathogenic or has a minimal pathogenic effect with said bacteria being either naturally occurring or genetically modified and is produced naturally, synthetically, or semi-synethically. Bactofection has been reported with, for example, species of Shigella, Salmonella, Listeria, and Escherichia coli., with results suggesting that bactofection can be used with any bacterial species (Weiss et al., Curr Opin Biotechnol. October 2001; 12(5):467-72).

Protein Delivery

In certain embodiments, the present invention relates to the delivery of an amino acid sequence of the invention conjugated to, fused with, or otherwise combined with, a peptide known as protein transduction domain (PTP). In particular embodiment, an amino acid sequence of the invention is the amino acid sequence for RNAse-L or a functional part thereof. A PTD is a short peptide that facilitates the movement of an amino acid sequence across an intact cellular membrane wherein said amino acid sequence would not penetrate the intact cellular membrane without being conjugated to, fused with, or otherwise combined with a PTD. The conjugation with, fusion to, or otherwise combination of a PTD with a heterologous molecule (including, for example, an amino acid sequence, nucleic acid sequence, or small molecule) is sufficient to cause transduction into a variety of different cells in a concentration-dependent manner. Moreover, when drawn to the delivery of amino acids, it appears to circumvent many problems associated with polypeptide, polynucleotide and drug-based delivery. Without being bound by theory, PTDs are typically cationic in nature causing PTDs to track into lipid raft endosomes and release their cargo into the cytoplasm by disruption of the endosomal vesicle. PTDs have been used for delivery of biologically active molecules, including amino acid sequences (see, for example, Viehl C. T., et al., Ann. Surg. Oncol. 12:517-525 (2005); Noguchi H., et al., Nat. Med. 10:305-309 (2004); and Fu A. L., et al., Neurosci. Lett. 368:258-62 (2004); Del Gazio Moore et al., J Biol Chem. 279(31):32541-4 (2004); US Application Publication No. 20070105775). For example, it has been shown that TAT-mediated protein transduction can be achieved with large proteins such as beta-galactosidase, horseradish peroxidase, RNAase, and mitochondrial malate dehydrogenase, whereby transduction into cells is achieved by chemically cross-linking the protein of interest to an amino acid sequence of HIV-1 TAT (see, for example, Fawell et al., PNAS, 91:664-668 (1994); Del Gazio et al., Mol Genet Metab. 80(1-2):170-80 (2003)).

Protein transduction methods encompassed by the invention include an amino acid sequence of the invention conjugated to, fused with, or otherwise combined with, a PTD. In particular embodiments a PTD of the invention includes, for example, the PTD from human transcription factor HPH-1, mouse transcription factor Mph-1, Sim-2, HIV-1 viral protein TAT, Antennapedia protein (Antp) of Drosophila, HSV-1 structural protein Vp22, regulator of G protein signaling R7, MTS, polyarginine, polylysine, short amphipathic peptide carriers Pep-1 or Pep-2, and other PTDs known to one of ordinary skill in the art or readily identifiable to one of ordinary skill in the art (see, for example, US Application Publication No. 20070105775). One of ordinary skill in the art could routinely identify a PTD by, for example, employing known methods in molecular biology to create a fusion protein comprising a potential PTD and, for example, green fluorescent protein (PTD-GFP) and detecting whether or not GFP was able to transduce a cellular membrane of intact cells, which can be determined by, for example, microscopy and the detection of internal fluorescence. It is noted that the particular PTD is not limited by any of the foregoing and the invention encompasses any known, routinely identifiable, and after-arising PTD.

Methods of protein transduction are known in the art and are encompassed by the present invention (see, for example, Noguchi et al. (2006) Acta Med. Okayama 60: 1-11; Wadia et al. (2002) Curr. Opin. Biotechnol. 13:52-56; Viehl C. T., et al., Ann. Surg. Oncol. 12:517-525 (2005); Noguchi H., et al., Nat. Med. 10:305-309 (2004); and Fu A. L., et al., Neurosci. Lett. 368:258-62 (2004); Del Gazio Moore et al., J Biol Chem. 279(31):32541-4 (2004); US Application Publication No. 2007/0105775; Gump et al., Trends in Molecular Medicine, 13(10):443-448 (2007); Tilstra et al., Biochem Soc Trans. 35(Pt 4):811-5 (2007); WO/2006/121579; US Application Publication No. 2006/0222657). In certain embodiments, a PTD may be covalently cross-linked to an amino acid sequence of the invention or synthesized as a fusion protein with an amino acid sequence of the invention followed by administration of the covalently cross-linked amino acid sequence and the PTD or the fusion protein comprising the amino acid sequence and the PTD. In other embodiments, methods for delivering an amino acid sequence of the invention includes a non-covalent peptide-based method using an amphipathic peptide as disclosed by, for example, Morris et al. Nat. Biotechnol. 19:1173-1176 (2001) and U.S. Pat. No. 6,841,535, and indirect polyethylenimine cationization as disclosed by, for example, Kitazoe et al. J. Biochem. 137:643-701 (2005).

As a non-limiting illustration of a method of making a PTD fusion protein, an expression system that permits the rapid cloning and expression of in-frame fusion polypeptides using an N-terminal 11 amino acid sequence corresponding to amino acids 47-57 of TAT is used (see, for example, Becker-Hapak et al., Methods 24:247-56 (2001); Schwarze et al., Science 285:1569-72 (1999); Becker-Hapak and Dowdy, Protein Transduction: Generation of Full-Length Transducible Proteins Using the TAT System; Curr Protoc Cell Biol. 2003 May; Chapter 20:Unit 20.2). Using this expression system, cDNA of the amino acid sequence of interest is cloned in-frame with the N-terminal 6× His-TAT-HA encoding region in the pTAT-HA expression vector. The 6× His motif provides for the convenient purification of a fusion polypeptide using metal affinity chromatography and the HA epitope tag allows for immunological analysis of the fusion polypeptide. Although recombinant polypeptides can be expressed as soluble proteins within E. coli, TAT-fusion polypeptides are often found within bacterial inclusion bodies. In the latter case, these proteins are extracted from purified inclusion bodies in a relatively pure form by lysis in denaturant, such as, for example, 8 M urea. The denaturation aids in the solubilization of the recombinant polypeptide and assists in the unfolding of complex tertiary protein structure which has been observed to lead to an increase in the transduction efficiency over highly-folded, native proteins (Becker-Hapak et al., supra). This latter observation is in keeping with earlier findings that supported a role for protein unfolding in the increased cellular uptake of the TAT-fusion polypeptide TAT-DHFR (Bonifaci et al., Aids 9:995-1000 (1995)). It is thought that the higher energy of partial or fully denatured proteins may transduce more efficiently than lower energy, correctly folded proteins, in part due to increased exposure of the TAT domain. Once inside the cells, these denatured proteins are properly folded by cellular chaperones such as, for example, HSP90 (Schneider et al., Proc. Natl. Acad. Sci. USA 93:14536-41 (1996)). Following solubilization, bacterial lysates are incubated with NiNTA resin (Qiagen), which binds to the 6× His domain in the recombinant protein. After washing, proteins are eluted from the column using increasing concentrations of imidazole. Proteins are further purified using ion exchange chromatography and finally exchanged into PBS+10% glycerol by gel filtration.

In certain embodiments the invention encompasses administration of an amino acid sequence of the invention conjugated to, fused with, or otherwise combined with, a PTD. In other embodiments, the invention encompasses administration of a nucleic acid sequence of the invention conjugated to, fused with, or otherwise combined with, a PTD. Both, an amino acid sequence and a nucleic acid sequence can be transduced across a cellular membrane when conjugated to, fused with, or otherwise combined with, a PTD. As such, administration of an amino acid sequence and a nucleic acid sequence is encompassed by the present invention. Routes of administration of an amino acid sequence or nucleic acid sequence of the invention include, for example, intraarterial administration, epicutaneous administration, ocular administration (e.g., eye drops), intranasal administration, intragastric administration (e.g., gastric tube), intracardiac administration, subcutaneous administration, intraosseous infusion, intrathecal administration, transmucosal administration, epidural administration, insufflation, oral administration (e.g., buccal or sublingual administration), oral ingestion, anal administration, inhalation administration (e.g., via aerosol), intraperitoneal administration, intravenous administration, transdermal administration, intradermal administration, subdermal administration, intramuscular administration, intrauterine administration, vaginal administration, administration into a body cavity, surgical administration (e.g., at the location of a tumor or internal injury), administration into the lumen or parenchyma of an organ, or other topical, enteral, mucosal, or parenteral administration, or other method, or any combination of the forgoing as would be known to one of ordinary skill in the art (see, for example, Remington's Pharmaceutical Sciences, 18th Ed. Mack Printing Company, 1990, incorporated herein by reference).

RNase-L activity

RNase L activity and expression is controlled by the 2′,5′-oligoadenylate synthetase. This enzyme is known to require dsRNA for activity, yet, to date, dsRNA has not been shown in any type of bacterial infection. Thus, it is quite surprising that a bacterial infection, which does not appear to involve dsRNA production, would somehow invoke the production and/or expresson of RNase-L in infected cells.

In certain embodiments, the invention is drawn to increasing the activity of RNase-L in cells harboring bacteria and/or bacterial spores. As described in the examples below and in the literature, 2′,5′-oligoadenylate synthetase produces 5′ phosphorylated, 2′,5′-linked oligoadenylates in response to IFN induction (Thakur et al., Proc Natl Acad Sci USA. 2007 Jun. 5; 104(23):9585-90. Epub 2007 May 29), which are collectively termed as 2-5A. 2-5A increases the activity of RNase-L (Id.; Zhou A, Hassel B A, Silverman R H. Cell. 1993; 72:753-765; Knight M, Cayley P J, Silverman R H, Wreschner D H, Gilbert C S, Brown R E, Kerr I M. Nature. 1980; 288:189-192). 2-5A has the general formula: px5′A(2′p5′A)n where x is about 1-3 and n is at least 2. In certain embodiments, 2-5A is the trimeric form.

In certain embodiments, the invention is drawn to increasing the activity of RNase-L by administering a small molecule. Small molecules that increase the activity of RNase-L, include, for example, C-5966451, C-5950331, C-5972155, C-5947495, C-6131864, C-6131645, C-6131416, C-6645744, C-6474572, C-5142087, and C-5973265 (Thakur et al., Proc Natl Acad Sci USA. 2007 Jun. 5; 104(23):9585-90. Epub 2007 May 29; Thakur et al., FASEB J. 2006 20:A74). See structures below.

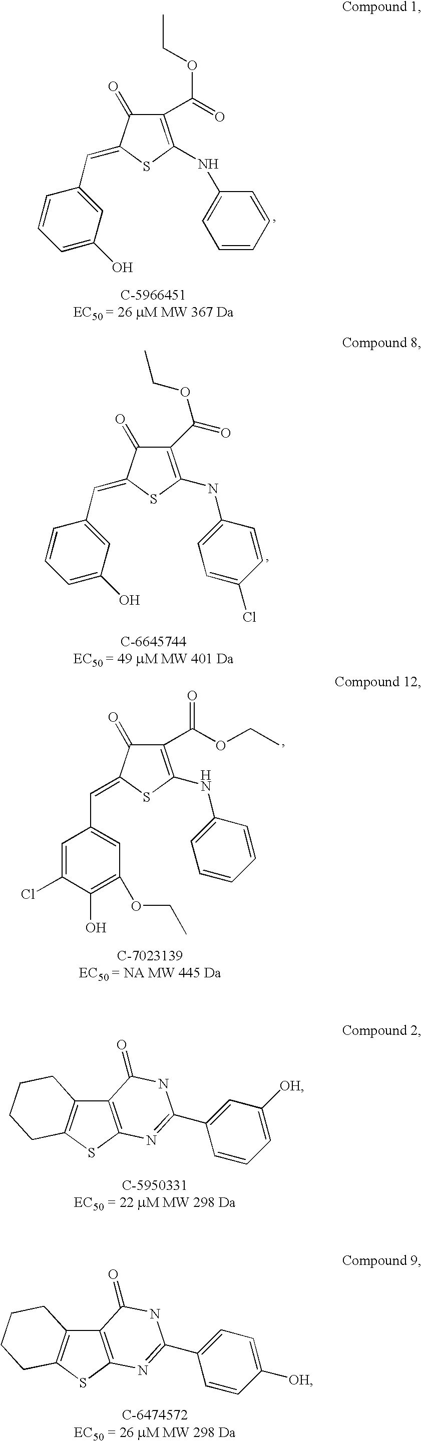

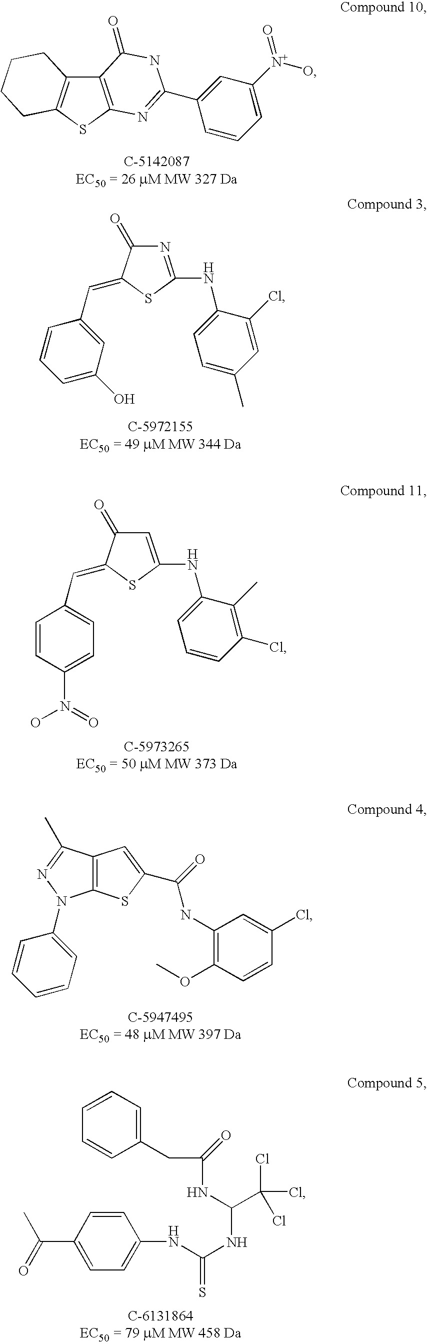

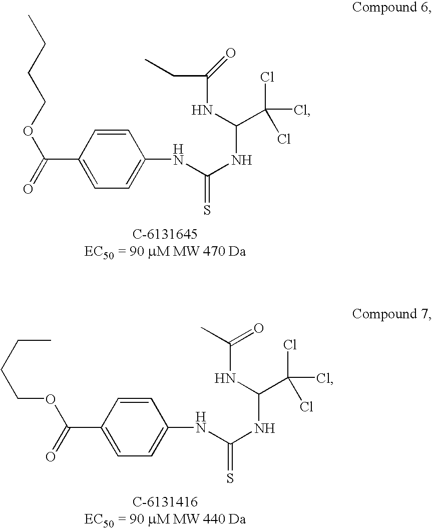

It is readily apparent to one of ordinary skill in the art, in light of teachings disclosed herein, that RNase-L activity plays an integral role in the function of the immune system. As taught herein, increases in RNase-L activity or increases in RNase-L expression are employed to effectively treat a microbial infection. It is also appreciated that increases in activity may result in untoward pathological effects resulting in an immune related disease or disorder.

An “immune related disease or disorder” refers to a disease or disorder wherein the immune system is enhanced or in which a component of the immune system causes, mediates or otherwise contributes to morbidity or morality. Also included is a disease or disorder in which depressing the immune response has an ameliorative effect on progression of the immune related disease or disorder. Included within an immune related disease or disorder is, for example, immune-mediated inflammatory diseases, inflammatory pain, non-immune-mediated inflammatory diseases, immunodeficiency diseases, cancer, etc., including, for example, celiac disease, inflammatory conditions of the lungs, systemic lupus erythematosis, discoid lupus erythematosus, subacute cutaneous lupus erythematosus, drug-induced lupus erythematosus, lupus nephritis, neonatal lupus, amyotrophic lateral sclerosis, rheumatoid arthritis, juvenile chronic arthritis, spondyloarthropathies, systemic sclerosis (e.g., scleroderma), idiopathic inflammatory myopathies (e.g., dermatoinyositis, polymyositis), Sjogren's syndrome, sarcoidosis, autoimmune hemolytic anemia (e.g., immune pancytopenia, paroxysmal nocturnal hemoglobinuria), autoimmune thrombocytopenia (e.g., idiopathic thrombocytopenic purpura, immune-mediated thrombocytopenia), thyroiditis (e.g., Grave's disease, Hashimoto's thyroiditis, juvenile lymphocytic thyroiditis, atrophic thyroiditis), diabetes mellitus, immune-mediated renal disease (e.g., glomerulonephritis, tubulointerstitial nephritis), demyelinating diseases of the central and peripheral nervous systems (e.g., multiple sclerosis), idiopathic demyelinating polyneuropathy or Guillain-Barre syndrome, multiple myositis, mixed connective tissue disease, hyperthyroidism, myasthenia gravis, autoimmune hepatopathy, ulcerative colitis, autoimmune nephropathy, autoimmune hematopathy, idiopathic interstitial pneumonia, hypersensitivity pneumonitis, autoimmune dermatosis, autoimmune cardiopathy, osteoarthritis, ARDS, interstitial cystitis, periodontitis/gingivitis, autoimmune infertility, Behcet's disease, chronic inflammatory demyelinating polyneuropathy, hepatobiliary diseases (e.g., infectious hepatitis and other non-hepatotropic viruses), autoimmune chronic active hepatitis, primary biliary cirrhosis, granulomatous hepatitis, and sclerosing cholangitis, inflammatory bowel disease (e.g., ulcerative colitis, Crohn's disease), gluten-sensitive enteropathy, Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease, X-linked infantile hypogammaglobulinemia, polysaccaride antigen unresponsiveness, transient hypogammaglobulinemia of infancy, and ankylosing spondylitis.

There is a biological balance concerning RNase-L activity such that not enough activity can result in a microbial infection or susceptibility to a microbial infection and too much activity can result in pathological effects resulting in, for example, an immune related disease or disorder or susceptibility to an immune related disease or disorder. For example, overexpression of RNase-L or high intracellular concentration of its activator results in apoptotic cell death (see, for example, Castelli et al., J Exp Med. 1997 Sep. 15; 186(6):967-72). Therefore, in certain embodiments, the invention is drawn to a method of treating an immune related disease or disorder in a subject in need thereof comprising administering an agent that decreases the activity of RNase-L. In other embodiments, the invention is drawn to a method of treating an immune related disease or disorder in a subject in need thereof comprising administering an agent that decreases the expression of RNase-L. Agents are of the same type as those described previously (i.e., an “agent” is a molecular entity including, for example, a small molecule, nucleic acid (such as, siRNA, shRNA expression cassette, antisense DNA, antisense RNA), protein, peptide, antibody, antisense drug, or other biomolecule that is naturally made, synthetically made, or semi-synthetically), except that the agent is directed to decreasing the activity of RNase-L and/or decreasing the expression of RNase-L as opposed to increasing the activity and/or expression of RNase-L as is the case for treating a microbial infection.

Mechanisms of decreasing the activity or expression of RNase-L can be achieved by, for example, small molecule antagonists of RNase-L; antibody, antibody fragments, and antibody fusion proteins directed RNase-L; nucleic acids (including, for example, 2-5A molecules and 2-5A analogues such as, for example, de-phosphorylated trimer, A2′p5′A2′p5′A [see, for example, Thakur et al., Proc Natl Acad Sci USA. 2007 Jun. 5; 104(23):9585-90. Epub 2007 May 29; Dong et al., J Biol Chem. 1994; 269:14153-14158), and 2-5An (see, for example, Bisbal et al, Biochemistry, 1987 Aug. 11; 26(16):5172-8; Bisbal et al., JBC 1995, Volume 270, Number 22, Issue of June 2, pp. 13308-13317, 1995); RLI (RNase L inhibitor) (see, for example, Mol. Cell. Biol., July 2000, vol. 20(14): 4959-4969; and silencing or interfering RNA.

In certain embodiments, the invention is drawn to decreasing the expression of RNase-L by utilizing silencing or interfering RNA. For example, double-stranded RNA is used as an interference molecule, e.g., RNA interference (RNAi), to decrease the expression of RNase-L. RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000), although such transfection or recombinant techniques as taught herein and is known by those of ordinary skill in the art can be used to delivery RNAi.

Another type of RNAi is often referred to as small interfering RNA (siRNA), which may also be utilized to decrease the expression of RNase-L. A siRNA may comprises a double stranded structure or a single stranded structure, the sequence of which is “substantially identical” to at least a portion of the target gene (See WO 04/046320, which is incorporated herein by reference in its entirety). “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated (see, for example: Computational Molecular Biology, Lesk, A. M., Oxford University Press, New York, 1988; Biocomputing: Informatics and Genome Projects, Smith, D. W., Academic Press, New York, 1993, and the methods disclosed in WO 99/32619, WO 01/68836, WO 00/44914, and WO 01/36646, all of which are specifically incorporated herein by reference). While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al.), BLASTP, BLASTN, and FASTA (Atschul et al.,) and CLUSTAL (Higgins et al., 1992; Thompson, et al., 1994).

Thus, siRNA contains a nucleotide sequence that is substantially identical to at least a portion of the target gene, for example, RNase-L, or any other molecular entity associated with RNase-L activity. One of skill in the art is aware that the nucleic acid sequences for RNase-L are readily available in GenBank, for example, GenBank accession NM_{—}021133, which is incorporated herein by reference in its entirety. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene (e.g., RNase-L), although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

There are several methods for preparing siRNA, such as chemical synthesis, in vitro transcription, siRNA expression vectors, and PCR expression cassettes. Irrespective of which method one uses, the first step in designing an siRNA molecule is to choose the siRNA target site, which can be any site in the target gene. In certain embodiments, one of skill in the art may manually select the target selecting region of the gene, which may be an ORF (open reading frame) as the target selecting region and may preferably be 50-100 nucleotides downstream of the “ATG” start codon. However, there are several readily available programs available to assist with the design of siRNA molecules, for example siRNA Target Designer by Promega, siRNA Target Finder by GenScript Corp., siRNA Retriever Program by Imgenex Corp., EMBOSS siRNA algorithm, siRNA program by Qiagen, Ambion siRNA predictor, Whitehead siRNA prediction, and Sfold. Thus, it is envisioned that any of the above programs may be utilized in the design and production of siRNA molecules that can be used in the present invention.

In certain embodiments, a method of treating an immune related disease or disorder in a subject in need thereof comprising administering an agent that decreases the activity or expression of RNase-L is administered prior to, concurrently with, or following the administration of one or more immune modulating molecules. An immune modulating molecule includes, for example, A-Hydrocort, A-Methapred, Aristospan, Betamethasone, Celestone, Cenocort, Cortef, Cortisone, Depo-Medrol, Hydrocortisone, Kenalog, Key-Pred, Medrol, Methylpred, Methylprednisolone, Orapred, Pediapred, Predicort, Prednisolone, Prednisone, Prelone, Sterapred, Triam, Triamcinolone, Acthar, Corticotropin, Dexamethasone, Azasan, Azathioprine, Imuran, Levothroid, Levothyroxine, Levoxyl, Synthroid, Unithroid, Carimune, Chlorambucil, Flebogamma, Gammagard, Immune Globulin (Human) (IGG), Iveegam, Leukeran, Octagam, Panglobulin, Polygam, Venoglobulin-S, Avonex, Betaseron, Interferon Beta-1a, Interferon Beta-1b, Rebif, Cyclophosphamide, Cytoxan, Neosar, CellCept, Mycophenolate Mofetil, Hydroxychloroquine, Plaquenil, Aralen, Chloroquine Phosphate, Thalidomide, Thalomid, Dapsone, Methotrexate, Rheumatrex, Trexall, Rilutek, Abatacept, Actron, Adalimumab, Amigesic, Anakinra, Naproxen, Ansaid, Arava, Ibuprofen, Aspirin, Auranofin, Azulfidine, Cataflam, Celebrex, Choline and Magnesium Salicylate, Clinoril, Cuprimine, Cyclosporine, Diflunisal, Etanercept, Etodolac, Fenoprofen, Flurbiprofen, Gold Sodium Thiomalate, Indomethacin, Infliximab, Ketoprofen, Lansoprazole-Naproxen, Leflunomide, Magnesium Salicylate/Phenyltoloxamine, Meclofenamate, Meloxicam, Nabumetone, oxoprozin, Penicillamine, Piroxicam, PREVACID NapraPAC, Rituximab, Pilocarpine, Salsalate, Sulfazine, Tolmetin, Azulfidine, Cleeravue-M, Dynacin Oral, Minocin, Interferon Alfa-n3, Budesonide, Mesalamine, Alkabel-SR, Anaspaz, Anti-Spas, Antispasmodic, A-Spas SL, Atropine, Atropine-Hyoscyamine-Scopolam, Belladonna Alkaloids, Belladonna Alk-Phenobarbital, BELLATAL ER, Bentyl, Clidinium-Chlordiazepoxide, Colidrops, Colytrol, Cystospaz, Dicyclomine, Dispas Chewable Melt, Donnamar, Donnatal, Hyoscyamine, Hyosophen, Hyospaz, Hyosyne, IB-Stat, Lahey Mixture #3, Levbid, Levsin, Levsinex, Librax (with Clinidium), Mar-Spas Chewable Melt, NuLev, Pahomin, Phenobarb-Belladonna Alkaloids, PRO-HYO Chewable Melt, Sal-Tropine, Simetyl, Simple Throat Irritations, Spacol, Symax, Acidophilus, Bacid, Cantil, Citrucel, Dairycare, Dofus, Enterogenic Concentrate, Equalactin, Fiber, Floranex, Flora-Q, Freeze Dried Acidophilus, GenFiber, Glycopyrrolate, Hydrocil, Intestinex, Konsyl Effervescent, Lactinex, Lactobac Ac& Pc-S.Therm-B.Anim, Laxate, Laxmar, M.F.A., Maldemar, Medi-Mucil, Mepenzolate Bromide, MetaFiber, Modane, Novaflor, Octreotide Acetate, Perdiem, Polycarbophil Calcium, Pro-Banthine, Pro-Bionate-C, Pro-Bionate-P, Propantheline, Psyllium Effervescent, Psyllium, Reguloid, Robinul, Sandostatin, Scopace, Scopolamine, Senna Prompt, Sennosides-Psyllium, Serutan, Smooth NVP, Superdophilus, V-Lax, Muromonab CD3, Orthoclone OKT3, tacrolimis, Drotrecogin, or other immune modulating molecules, including, for example, anti-inflammatories. In further particular embodiments, an immune modulating molecule comprises two or more of the foregoing immune modulating molecules.

While the invention has been described with reference to certain particular embodiments thereof, those skilled in the art will appreciate that various modifications may be made without departing from the spirit and scope of the invention. The scope of the appended claims is not to be limited to the specific embodiments described.

### Examples

### Example 1

### RNase-L−/− Mice Exhibit Reduced Survival and Microbiocidal Activity in Response to B. anthracis and E. coli Challenge

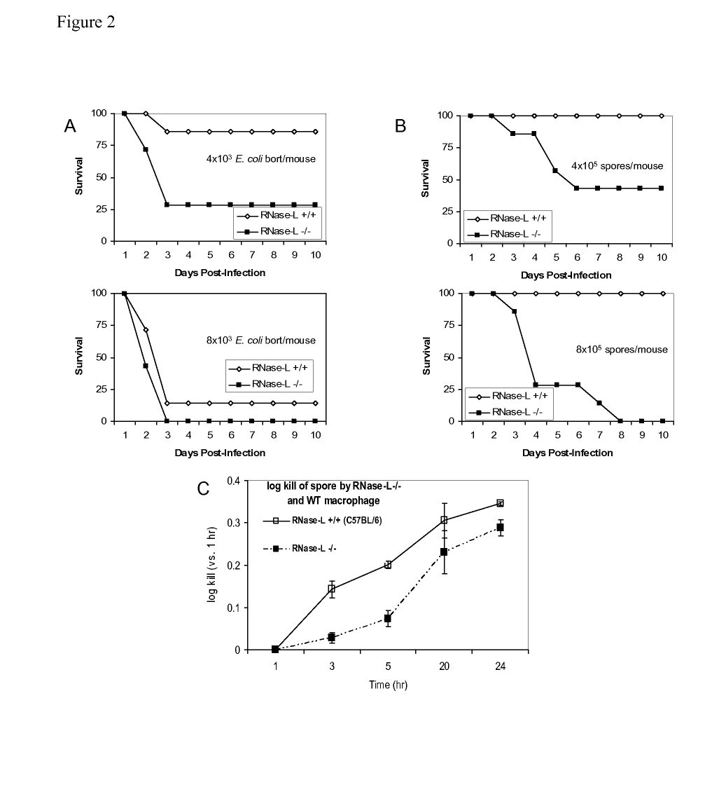

Type 1 IFNs are an essential component of the innate immune response (Karaghiosoff et al. (2003) Nature immunology 4, 471-477; Toshchakov et al. (2002) Nature immunology 3, 392-398; Basu et al. (2007) Infection and immunity 75, 2351-2358). RNase-L is a mediator of IFN-induced antiviral and antiproliferative activities. Under these premises RNase-L was tested as an antimicrobial against gram-positive and gram-negative bacteria, B. anthracis (BA) and E. coli respectively, which are important human pathogens. RNase-L −/− and wild type C57B1/6 mice (WT) were injected intraperitoneally (IP) with two doses of BA Stern 34F2 spores, an attenuated BA variant that is defective in capsule production, or E. coli bort strain, and monitored for signs of disease and survival. Remarkably, RNase-L −/− mice exhibited a significantly increased mortality in response to both microbes, as compared to WT mice (FIG. 2). Whereas the WT mice did not succumb to BA infection during the course of the experiment, 86% of the RNase-L −/− mice died by four days of infection with the high BA dose, and 100% had died by eight days of infection. Similarly, RNase-L −/− mice exhibited a markedly enhanced susceptibility to E. coli challenge at two separate infectious doses. These findings identify a significant, and previously unrecognized, role for RNase-L in the host immune response to a microbial pathogens.

Macrophages play critical role in the early host response to pathogens. Therefore, we determined if the reduced survival of RNase-L −/− mice to microbial challenge reflected compromised microbiocidal activity in RNase-L −/− macrophages. In fact, BA spore killing was dramatically reduced in RNase-L −/− as compared to WT macrophages at 5 hpi (FIG. 2C). However, the production of NO, an important mediator of microbiocidal activity, did not differ between the RNase-L −/− and WT macrophages, suggesting that RNase-L does not impact this component of microbiocidal action. Thus, the reduced survival of RNase-L −/− mice to challenge with BA spores corresponds to an early defect in microbiocidal activity.

### Example 2

### Microbial Load is Increased, Induction of Proinflammatory Cytokines is Decreased, and the Immune Cell Profile is Altered following E. coli Challenge of RNase-L −/− Mice

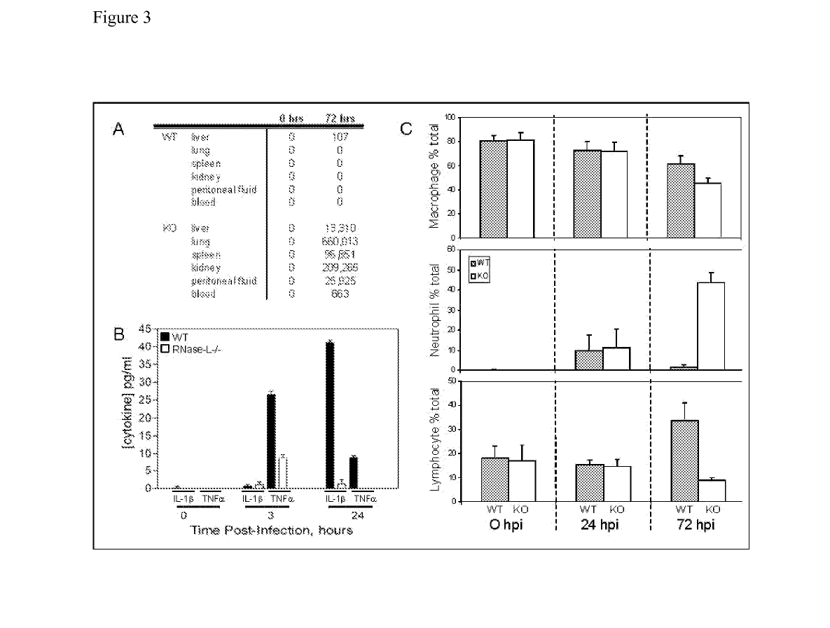

The increased mortality of RNase-L −/− mice following microbial challenge may reflect an impaired immune response resulting in increased microbial load, or may be due to the dysregulated overproduction of host proinflammatory mediators. To identify differences in the pathogenesis of E. coli infection in RNase-L −/− and WT mice, the measurement of microbial load, proinflammatory cytokine induction, and the profile of peritoneal immune cell infiltrates following infection with E. coli was carried out. Specifically, microbes were quantified in liver, lung, kidney, spleen, blood, and peritoneal fluid, and the expression of IL-1β and TNFα in the plasma was determined by ELISA at various post-infection time points. The microbial load was small, and did not dramatically differ between RNase-L −/− and WT mice at early time points. However, by 72 hpi, a significant increase in microbial load was observed in most tissues of RNase-L −/− as compared to WT mice (FIG. 3A). The increased microbial load corresponded with a dramatically diminished induction of plasma IL-1β and TNFα at early times post-infection in RNase-L −/− mice (FIG. 3B). Quantification of peritoneal immune cell infiltrates revealed further striking differences between RNase-L −/− and WT mice. There were no significant differences in the numbers of macrophages, neutrophils, or lymphocytes between RNase-L −/− and WT mice in the absence of infection. However, a dramatic increase in the number of neutrophils, and to a lesser extent, macrophages, in the RNase-L −/− mice was observed by 72 hpi (FIG. 3C). In contrast, a concomitant decrease in the relative lymphocyte population was observed in RNase-L −/− mice. Thus, the recruitment or trafficking of immune cells is markedly altered in RNase-L −/− mice. These findings indicate that multiple components of the host immune response are compromised in RNase-L −/− mice, and that the impaired capacity of RNase-L −/− mice to clear a microbial infection, rather than the potential overproduction of host cytokines, is responsible for the increased mortality observed.

### Example 3

### RNase-L-Dependent Gene Expression in BA Infected Macrophages

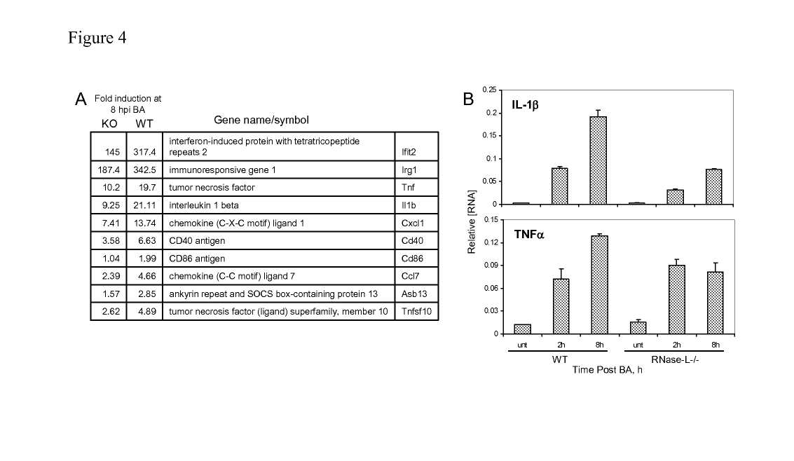

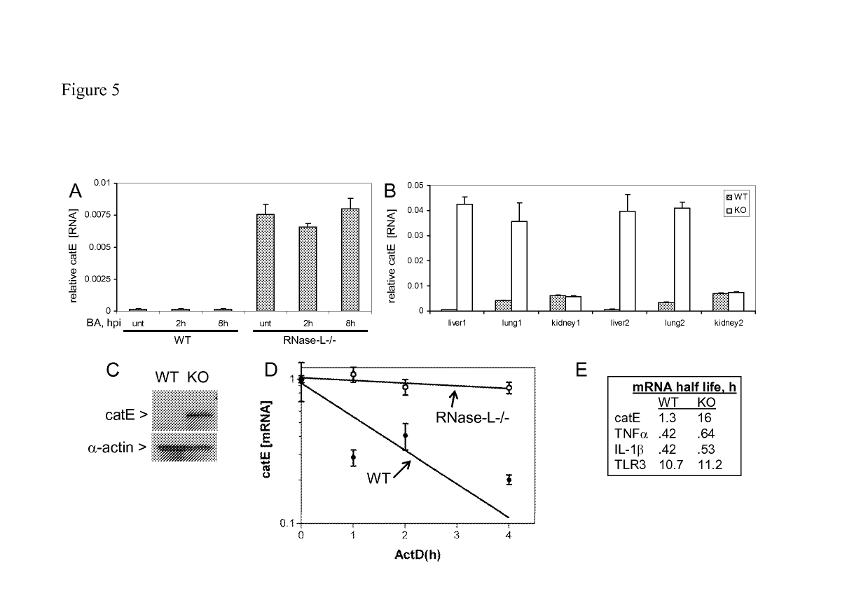

The diminished induction of cytokines and impaired recruitment of inflammatory effector cells in response to E. coli infection of RNase-L −/− mice suggested that altered host gene expression may account, in part, for the increased susceptibility of these mice to microbial challenge. To identify RNase-L-dependent changes in host gene expression that may mediate its antimicrobial activity, we performed a microarray analysis of WT and RNase-L −/− macrophages following infection with BA spores for two and eight hours. Microarray analysis was performed on triplicate samples using an affymetrix chip containing >45,000 probe sets representing ˜34,000 mRNAs (Virginia Bioinformatics Institute). The data was filtered to identify transcripts for which the BA-induced change in expression differed by +/−1.75 fold or greater between RNase-L −/− and WT macrophages. Thirty-four unique genes met these criteria, ten of which encoded multiple classes of proteins associated with immune functions (FIG. 4A). Most notably, the induction of the proinflammatory cytokines IL-1β and TNFα, which play critical roles in the early host response to BA spores (Basu et al. (2007) Infection and immunity 75, 2351-2358), was significantly diminished in the RNase-L −/− macrophages, and this altered expression was validated by qPCR (FIG. 4B). In contrast, the induction of other primary response antimicrobial genes, such as IFNβ, was equivalent in RNase-L −/− and WT macrophages (not shown). These findings agree well with the reduced levels of these cytokines in the plasma of RNase-L −/− mice following E. coli challenge (FIG. 3B), suggesting that the early response to BA and E. coli involves similar pathways. Consistent with the striking differences observed in immune cell infiltrates following infection of RNase-L −/− and WT mice (FIG. 3C), the expression of several chemokines that play important roles as chemoattractants to recruit immune effector cells to sites of inflammation (e.g. CXCL1/GRO1, CCL7/MCP3) was reduced in RNase-L −/− macrophages (FIG. 4A). These data indicate that RNase-L is required for the optimal induction of a subset of immune response genes in response to BA spores and that the impaired induction of these genes may underlie the enhanced susceptibility of RNase-L −/− mice to BA microbial challenge.

### Example 4

### Cathepsin E is a Primary Target of RNase-L Regulation in Macrophages

RNase-L can downregulate gene expression through degradation of substrate mRNAs and can induce gene expression via secondary, indirect effects of substrate degradation (e.g. if an RNase-L mRNA substrate encodes a transcriptional repressor). The expression of RNase-L substrates is predicted to be enhanced in RNase-L −/− macrophages. However, our microarray analysis revealed that immune response genes were downregulated in RNase-L −/− macrophages, suggesting that RNase-L indirectly modulated their expression. To identify the direct mRNA targets of RNase-L in macrophages, the degradation of which may be required for antimicrobial activities including the induction of proinflammatory cytokines, we analyzed the microarray data for transcripts that were upregulated in RNase-L −/− macrophages and represented candidate RNase-L substrates. This analysis revealed that the mRNA for cathepsin E (catE) was upregulated by 20 fold in basal, and BA-infected, RNase-L −/− macrophages. Remarkably, only four other transcripts represented on the array were upregulated in RNase-L −/− macrophages and the magnitude of their upregulation was approximately 10-fold less than that observed for catE (not shown). This finding suggested that catE mRNA is a primary substrate of RNase-L in macrophages. The RNase-L-dependent regulation of catE mRNA identified in the microarray analysis was validated by qPCR (FIG. 5A). Interestingly, these results clearly indicate that RNase-L regulates basal catE expression, independent of microbial infection or induction of IFN. However, basal RNase-L expression is dependent on constitutively expressed IFNβ in macrophages, which may be an important factor in catE regulation (Thomas et al. (2006) J Biol Chem. 281(41):31119-30). Thus in this context, RNase-L serves to maintain catE expression at a level that is required for an optimal response to microbial challenge. Elevated catE expression was also observed in liver and lung tissues from RNase-L −/− mice suggesting that RNase-L-dependent regulation is not restricted to macrophages. In contrast, catE mRNA did not differ in RNase-L −/− and WT kidney or spleen tissues in which expression was uniformly low and high respectively (FIG. 5B; spleen expression is >7-fold that of other tissues, not shown). Thus, the regulation of catE by RNase-L may exhibit tissue specificity. Importantly, catE protein expression reflected the dramatic increase in catE mRNA in RNase-L −/− macrophages (FIG. 5C).

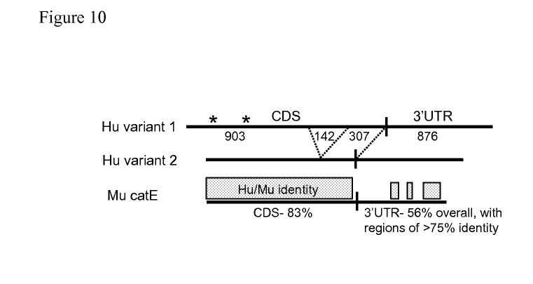

If catE mRNA is a substrate of RNase-L, the increase in steady state catE mRNA observed in RNase-L −/− macrophages is predicted to correspond to an increase in catE mRNA stability. In fact, analysis of catE mRNA stability following transcriptional arrest by actinomycin-D revealed a dramatic, 12-fold increase in the catE mRNA half-life in RNase-L −/− macrophages as compared to that in WT macrophages (FIG. 5D). In contrast, the half-life of other cellular mRNAs encoding unstable (TNFα, IL-1β), and stable (TLR3), mRNAs were slightly elevated in RNase-L −/− macrophages, but did not differ greatly (FIG. 5E). Consistent with status of catE mRNA as an RNase-L substrate, two putative RNase-L recognition sites were identified at positions 286-314 and 602-632 in the catE transcript (FIG. 10).

This motif was previously identified using the FOLDALIGN program to search for sequence and structural RNA motifs among nonaligned sequences of mRNAs that were downregulated following RNase-L activation in WI38 human fibroblasts, and represented candidate RNase-L substrates (unpublished data). The 34 base motif that contained a high frequency of UA and UU doublets as expected for an RNase-L cleavage site (Wreschner et al. (1981) Nature 289, 414-417). As further confirmation of the substrate status of catE mRNA, the physical association of catE mRNA with a RNase-L complex in cells are analyzed. Thus, catE mRNA represents a novel RNase-L-regulated transcript that is selectively targeted for degradation in macrophages.

### Example 5

### RNase-L-Dependent Regulation of catE-Mediated Activities: a Potential Mechanism for the Antimicrobial Action of RNase-L

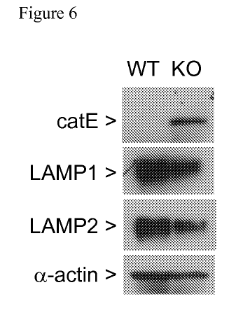

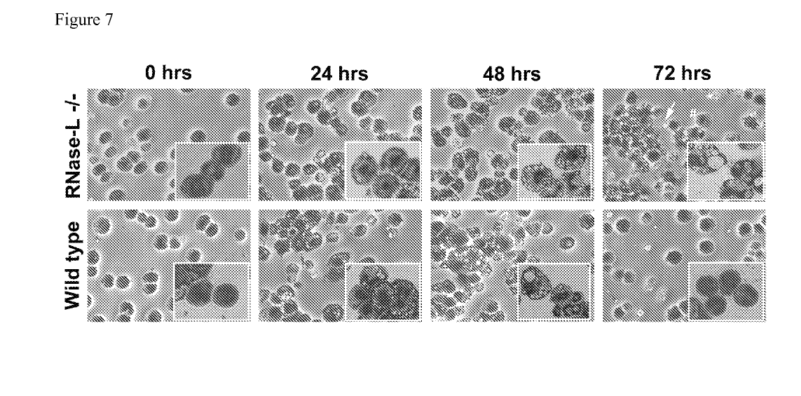

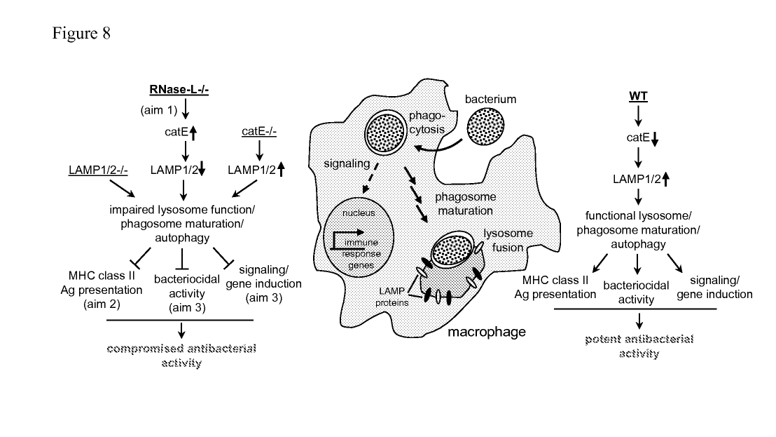

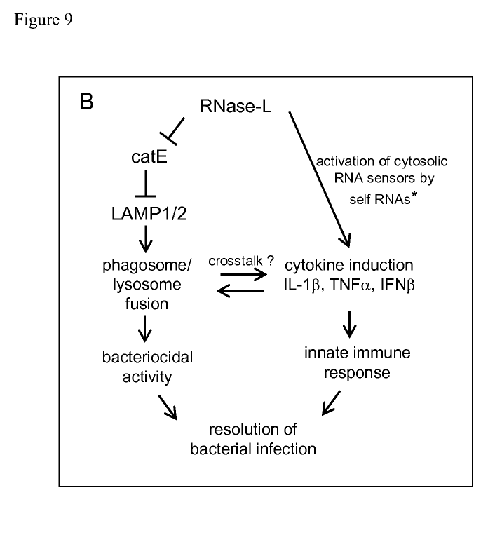

CatE is an aspartic proteinase that mediates multiple immune functions as a component of the endolysosomal pathway (Yanagawa et al. (2007) J Biol Chem 282, 1851-1862; Tsukuba et al. (2003) J Biochem (Tokyo) 134, 893-902; Nishioku et al. (2002) J Biol Chem 277, 4816-4822; Chain et al. (2005) J Immunol 174, 1791-1800; Tsukuba et al. (2006) J Biochem (Tokyo) 140, 57-66 Yanagawa et al. (2007) J Biol Chem 282, 1851-1862; Tsukuba et al. (2003) J Biochem (Tokyo) 134, 893-902; Nishioku et al. (2002) J Biol Chem 277, 4816-4822; Chain et al. (2005) J Immunol 174, 1791-1800; Tsukuba et al. (2006) J Biochem (Tokyo) 140, 57-66). In light of the RNase-L-dependent regulation of catE, catE-mediated functions were predicted to contribute to the antimicrobial activity of RNase-L. An important component of this prediction is that catE substrates will be downregulated in RNase-L −/− macrophages, as a result of the increased expression of endogenous catE in these cells. Consistent with this, expression of the catE substrates LAMP 1 and 2 were downregulated in RNase-L −/− macrophages (FIG. 6). In addition, the induction of IL-1β, thought to be an extracellular catE substrate, is significantly diminished in RNase-L −/− macrophages (FIG. 3B). Thus, the increase in catE protein in RNase-L−/− macrophages results in a corresponding increase in its functional activity. The upregulation of LAMP 1/2 proteins is implicated in the altered lysosome-associated immune functions observed in catE −/− macrophages (Yanagawa et al. (2007) J Biol Chem 282, 1851-1862). Interestingly, LAMP 1/2 −/− cells also exhibit defects in lysosome function (Huynh et al. (2007) Embo J 26, 313-324). Taken together, these findings indicate that dysregulated expression of LAMP 1/2 proteins, either upregulated, as in catE −/− macrophages, or abrogated, as in LAMP1/2 −/− macrophages, results in a functional disruption of endolysosomal activities. The endolysosomal pathway is essential for host antimicrobial activities including the elimination of microbes in phagosomes and autophagosomes, and the processing and presentation of antigens in association with MHC class II molecules. Therefore, we postulated that the decrease in LAMP 1/2 expression in RNase-L −/− macrophages will result in impaired endolysosomal functions, and that this defect may contribute to the compromised antimicrobial response observed in these cells. Importantly, a relatively modest downregulation of LAMP1/2 expression, similar to that observed in RNase-L −/− macrophages, was previously shown to functionally modulate lysosome activity (Huynh et al. (2007) Embo J 26, 313-324). Thus, the altered expression of LAMP1/2 may also impact lysosome function in RNase-L −/− macrophages. Consistent with this, macrophages in peritoneal infiltrates of E. coli infected RNase-L −/− mice exhibited an enlarged, highly vacuolated morphology at later times of infection when WT macrophages had returned to a quiescent morphology, indiciating that RNase-L −/− macrophages could not process phagocytosed cargo. WT macrophages displayed a vacuolated appearance at 24 and 48 hpi, which is characteristic of phagocytic activity (FIG. 7). However, by 72 hpi the size and vacuolization of WT macrophages was dramatically reduced and resembled that of macrophages from uninfected mice. In striking contrast, RNase-L −/− macrophages did not appear activated until 48 hpi and retained the high degree of vacuolization through 72 hpi. These data demonstrate that RNase-L −/− macrophages are capable of internalizing microbes, but have a defect in one or more steps of phagosome maturation. This defect may reflect the altered regulation of LAMP1/2 and resultant impairment of lysosome function.

Interestingly, recent studies determined that TLR signaling pathways induced by microbial pathogens are required for phagosome maturation (Blander et al. (2006) Nature immunology 7, 1029-1035; Blander et al. (2004) Science 304, 1014-1018). In light of this finding, the impaired lysosome function observed in catE −/− macrophages, and in RNase-L−/− macrophages, may be linked to the diminished induction of proinflammatory cytokines in both of these systems (FIGS. 3B and 4B).

Taken together, these data support a model in which the RNase-L-dependent regulation of catE is an important component of the host antimicrobial response (FIG. 8). Furthermore, these data support exploiting the innate immune response and RNase-L, and other associated protein, for the treatment of antimicrobial infections and associated conditions or diseases.

### Example 6

### RNase L and CatE Expression in Bacterial Infection

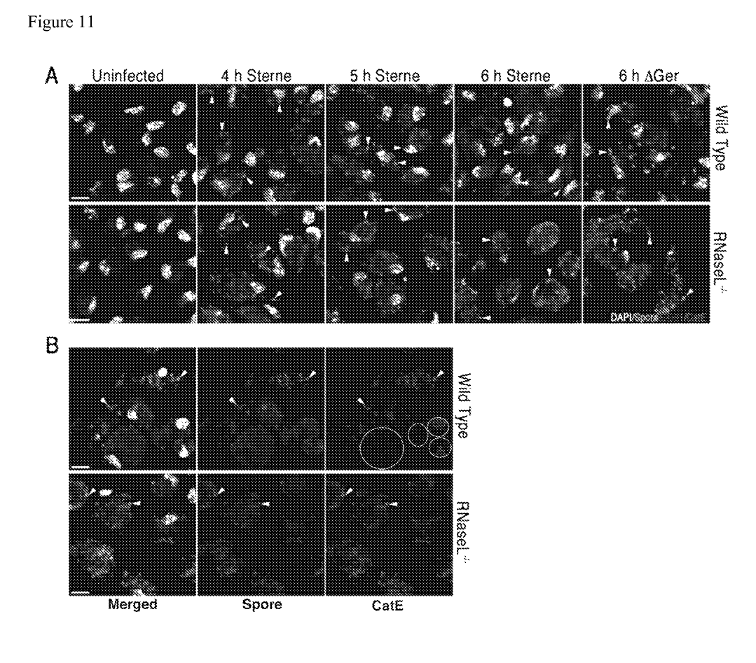

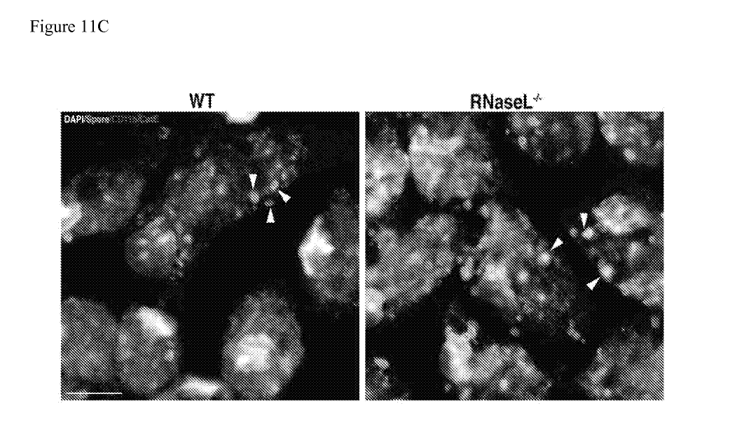

1. To monitor the fates of internalized bacteria in WT and RNase-L−/− macrophages directly, and to evaluate how this may relate to CatE expression, confocal microscopy was used to analyze BA infection of macrophages over time (FIG. 11). Staining for CatE confirmed its overexpression in RNase-L−/− macrophages, and revealed a cytoplasmic and perinulcear distribution, as compared to the exclusively perinuclear localization observed in WT macrophages (FIG. 11A, uninfected). Infection with Sterne and a germination-deficient BA strain (Δ-Ger) demonstrated that spores were internalized with equal efficiency in WT and RNase-L−/− macrophages, and co-localized with CatE prior to germination (arrowheads in 4 h Sterne and 6 h Δ-Ger of FIGS. 11A, and 11B). However, upon spore germination, the co-localization with CatE was lost in WT, but not RNase-L−/− macrophages (FIGS. 11A,B). This phenotype was more clearly visualized when signals from spores and CatE were viewed separately in identical fields (FIG. 11B). Upon spore germination in WT macrophages, the punctate, green spore signal became diffuse, due to the breakdown of the exosporium, and coincided with a complete loss of CatE co-localization (FIG. 11B, compare the co-localization signal in ungerminated spores identified by arrowheads, with that in the outlined macrophage post-germination, and see FIG. 11C). In contrast, CatE remained associated with spore components following germination in RNase-L−/− macrophages (compare WT and RNase-L−/− 6 h Sterne in FIGS. 11A and 11C). These observations identify a specific alteration in the host response to BA infection that is associated with CatE overexpression in RNase-L−/− macrophages, suggesting that the RNase-L-dependent regulation of CatE is critical for the proper processing and elimination of internalized BA. Indeed, exosporium breakdown is required for lysosome targeting and bactericidal activity, thus the protracted association of CatE with spore structures in RNase-L−/− macrophages may impede this process.

Importantly, the diminished induction of proinflammatory cytokines in RNase-L−/− mice corresponded with a marked delay in endotoxin-induced lethality, demonstrating the physiologic relevance of this phenotype (Table 1). Thus, similar to other host defense mediators (e.g. IFN3, 15; TLR416), the immune response defects in RNase-L−/− mice that are protective against a lethal, non-replicative insult (e.g., LPS), are associated with increased susceptibility to infection with a live bacterium (E. coli).

TABLE 1
Survival post LPS treatment

| | Day 1 | Day 2 | Day 3 |
|---|---|---|---|
| C57BL/6(WT) | 4/18 | 1/18 | 0/18 |
| RNase-L −/− | 12/19 | 1/19 | 0/19 |

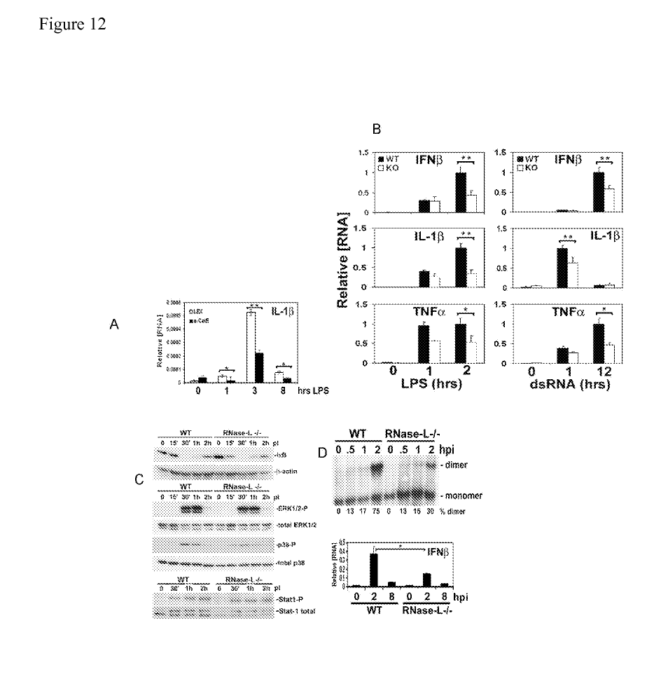

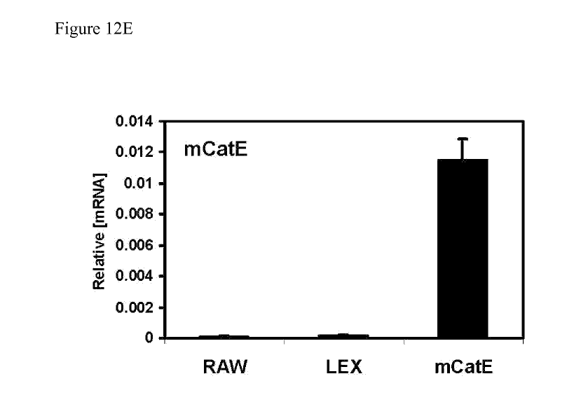

In light of this crosstalk, and to examine the potential role of CatE in RNase-L-regulated cytokine induction, we determined if CatE overexpression could mimic any components of the RNase-L−/− phenotype. Remarkably, similar to RNase-L−/− macrophages, induction of IL-1β was diminished following LPS treatment of CatE-transduced as compared to vector control RAW264.7 macrophages, thus linking CatE regulation by RNase-L to its impact on cytokine induction (FIG. 12A). However, ectopic expression of CatE did not alter LPS induction of TNFα, suggesting that RNase-L-mediated activities independent of CatE regulation are required.

References

All patents and publications mentioned in this specification and those listed below are indicative of the level of those of ordinary skill in the art to which the invention pertains. All patents and publications herein are incorporated by reference to the same extent as if each individual publication was specifically and individually indicated as having been incorporated by reference in its entirety. All of the following references and those cited throughout the specification are incorporated by reference in their entirety.

### REFERENCES

Stetson, D. B., and Medzhitov, R. (2006) Type I interferons in host defense, Immunity 25, 373-381.

Decker, T., Stockinger, S., Karaghiosoff, M., Muller, M., and Kovarik, P. (2002) IFNs and STATs in innate immunity to microorganisms, The Journal of clinical investigation 109, 1271-1277.

Akira, S. (2006) TLR signaling, Current topics in microbiology and immunology 311, 1-16.

Mariathasan, S., and Monack, D. M. (2007) Inflammasome adaptors and sensors: intracellular regulators of infection and inflammation, Nature reviews 7, 31-40.

Karaghiosoff, M., Steinborn, R., Kovarik, P., Kriegshauser, G., Baccarini, M., Donabauer, B., Reichart, U., Kolbe, T., Bogdan, C., Leanderson, T., Levy, D., Decker, T., and Muller, M. (2003) Central role for type I interferons and Tyk2 in lipopolysaccharide-induced endotoxin shock, Nature immunology 4, 471-477.

Stark, G. R., Kerr, I. M., Williams, B. R., Silverman, R. H., and Schreiber, R. D. (1998) How cells respond to interferons, Annu Rev Biochem 67, 227-264.

Carpten, J., Nupponen, N., Isaacs, S., Sood, R., Robbins, C., Xu, J., Faruque, M., Moses, T., Ewing, C., Gillanders, E., Hu, P., Bujnovszky, P., Makalowska, I., Baffoe-Bonnie, A., Faith, D., Smith, J., Stephan, D., Wiley, K., Brownstein, M., Gildea, D., Kelly, B., Jenkins, R., Hostetter, G., Matikainen, M., Schleutker, J., Klinger, K., Connors, T., Xiang, Y., Wang, Z., De Marzo, A., Papadopoulos, N., Kallioniemi, O. P., Burk, R., Meyers, D., Gronberg, H., Meltzer, P., Silverman, R., Bailey-Wilson, J., Walsh, P., Isaacs, W., and Trent, J. (2002) Germline mutations in the ribonuclease L gene in families showing linkage with HPC1, Nat Genet 30, 181-184.

Meurs, E. F., Galabru, J., Barber, G. N., Katze, M. G., and Hovanessian, A. G. (1993) Tumor suppressor function of the interferon-induced double-stranded RNA-activated protein kinase, Proc Natl Acad Sci USA 90, 232-236.

Silverman, R. H. (2003) Implications for RNase L in prostate cancer biology, Biochemistry 42, 1805-1812.

Wreschner, D. H., McCauley, J. W., Skehel, J. J., and Kerr, I. M. (1981) Interferon action—sequence specificity of the ppp(A2′p)nA-dependent ribonuclease, Nature 289, 414-417.

Benoit De Coignac, A., Bisbal, C., Lebleu, B., and Salehzada, T. (1998) cDNA cloning and expression analysis of the murine ribonuclease L inhibitor, Gene 209, 149-156.

Kubota, K., Nakahara, K., Ohtsuka, T., Yoshida, S., Kawaguchi, J., Fujita, Y., Ozeki, Y., Hara, A., Yoshimura, C., Furukawa, H., Haruyama, H., Ichikawa, K., Yamashita, M., Matsuoka, T., and Iijima, Y. (2004) Identification of 2′-phosphodiesterase, which plays a role in the 2-5A system regulated by interferon, J Biol Chem 279, 37832-37841.

Li, X. L., Blackford, J. A., and Hassel, B. A. (1998) RNase L mediates the antiviral effect of interferon through a selective reduction in viral RNA during encephalomyocarditis virus infection, J Virol 72, 2752-2759.

Bisbal, C., Silhol, M., Laubenthal, H., Kaluza, T., Carnac, G., Milligan, L., Le Roy, F., and Salehzada, T. (2000) The 2′-5′ oligoadenylate/RNase L/RNase L inhibitor pathway regulates both MyoD mRNA stability and muscle cell differentiation, Mol Cell Biol 20, 4959-4969.

Chandrasekaran, K., Mehrabian, Z., Li, X. L., and Hassel, B. (2004) RNase-L regulates the stability of mitochondrial DNA-encoded mRNAs in mouse embryo fibroblasts, Biochem Biophys Res Commun 325, 18-23.

Khabar, K. S., Siddiqui, Y. M., al-Zoghaibi, F., al-Haj, L., Dhalla, M., Zhou, A., Dong, B., Whitmore, M., Paranjape, J., Al-Ahdal, M. N., Al-Mohanna, F., Williams, B. R., and Silverman, R. H. (2003) RNase L mediates transient control of the interferon response through modulation of the double-stranded RNAdependent protein kinase PKR, J Biol Chem 278, 20124-20132.

Castelli, J. C., Hassel, B. A., Wood, K. A., Li, X. L., Amemiya, K., Dalakas, M. C., Torrence, P. F., and Youle, R. J. (1997) A study of the interferon antiviral mechanism: apoptosis activation by the 2-5A system, J Exp Med 186, 967-972.

Diaz-Guerra, M., Rivas, C., and Esteban, M. (1997) Activation of the IFN-inducible enzyme RNase L causes apoptosis of animal cells, Virology 236, 354-363.

Leitner, W. W., Hwang, L. N., deVeer, M. J., Zhou, A., Silverman, R. H., Williams, B. R., Dubensky, T. W., Ying, H., and Restifo, N. P. (2003) Alphavirus-based DNA vaccine breaks immunological tolerance by activating innate antiviral pathways, Nat Med 9, 33-39.

Silverman, R. H., Zhou, A., Auerbach, M. B., Kish, D., Gorbachev, A., and Fairchild, R. L. (2002) Skin allograft rejection is suppressed in mice lacking the antiviral enzyme, 2′,5′-oligoadenylate-dependent RNase L, Viral immunology 15, 77-83.

Malathi, K., Paranjape, J. M., Bulanova, E., Shim, M., Guenther-Johnson, J. M., Faber, P. W., Eling, T. E., Williams, B. R., and Silverman, R. H. (2005) A transcriptional signaling pathway in the IFN system mediated by 2′-5′-oligoadenylate activation of RNase L, Proc Natl Acad Sci USA 102, 14533-14538.

Yanagawa, M., Tsukuba, T., Nishioku, T., Okamoto, Y., Okamoto, K., Takii, R., Terada, Y., Nakayama, K. I., Kadowaki, T., and Yamamoto, K. (2007) Cathepsin E deficiency induces a novel form of lysosomal storage disorder showing the accumulation of lysosomal membrane sialoglycoproteins and the elevation of lysosomal pH in macrophages, J Biol Chem 282, 1851-1862.

Yasuda, Y., Tsukuba, T., Okamoto, K., Kadowaki, T., and Yamamoto, K. (2005) The role of the cathepsin E propeptide in correct folding, maturation and sorting to the endosome, J Biochem (Tokyo) 138, 621-630.

Cook, M., Caswell, R. C., Richards, R. J., Kay, J., and Tatnell, P. J. (2001) Regulation of human and mouse procathepsin E gene expression, Eur J Biochem 268, 2658-2668.

25. Yee, C. S., Yao, Y., Li, P., Klemsz, M. J., Blum, J. S., and Chang, C. H. (2004) Cathepsin E: a novel target for regulation by class II transactivator, J Immunol 172, 5528-5534.

Tsukuba, T., Okamoto, K., Okamoto, Y., Yanagawa, M., Kohmura, K., Yasuda, Y., Uchi, H., Nakahara, T., Fume, M., Nakayama, K., Kadowaki, T., Yamamoto, K., and Nakayama, K. I. (2003) Association of cathepsin E deficiency with development of atopic dermatitis, J Biochem (Tokyo) 134, 893-902.

Nishioku, T., Hashimoto, K., Yamashita, K., Liou, S. Y., Kagamiishi, Y., Maegawa, H., Katsube, N., Peters, C., von Figura, K., Saftig, P., Katunuma, N., Yamamoto, K., and Nakanishi, H. (2002) Involvement of cathepsin E in exogenous antigen processing in primary cultured murine microglia, J Biol Chem 277, 4816-4822.

Chain, B. M., Free, P., Medd, P., Swetman, C., Tabor, A. B., and Terrazzini, N. (2005) The expression and function of cathepsin E in dendritic cells, J Immunol 174, 1791-1800.

Tsukuba, T., Yamamoto, S., Yanagawa, M., Okamoto, K., Okamoto, Y., Nakayama, K. I., Kadowaki, T., and Yamamoto, K. (2006) Cathepsin E-deficient mice show increased susceptibility to bacterial infection associated with the decreased expression of multiple cell surface Toll-like receptors, J Biochem (Tokyo) 140, 57-66.

Blander, J. M., and Medzhitov, R. (2006) On regulation of phagosome maturation and antigen presentation, Nature immunology 7, 1029-1035.

Aderem, A., and Underhill, D. M. (1999) Mechanisms of phagocytosis in macrophages, Annual review of immunology 17, 593-623.

Eskelinen, E. L. (2006) Roles of LAMP-1 and LAMP-2 in lysosome biogenesis and autophagy, Molecular aspects of medicine 27, 495-502.

Huynh, K. K., Eskelinen, E. L., Scott, C. C., Malevanets, A., Saftig, P., and Grinstein, S. (2007) LAMP proteins are required for fusion of lysosomes with phagosomes, Embo J 26, 313-324.

Kirkegaard, K., Taylor, M. P., and Jackson, W. T. (2004) Cellular autophagy: surrender, avoidance and subversion by microorganisms, Nat Rev Microbiol 2, 301-314.

Menendez-Benito, V., and Neefjes, J. (2007) Autophagy in MHC class II presentation: sampling from within, Immunity 26, 1-3.

Blander, J. M., and Medzhitov, R. (2004) Regulation of phagosome maturation by signals from toll-like receptors, Science 304, 1014-1018.

Busquets, L., Guillen, H., DeFord, M. E., Suckow, M. A., Navari, R. M., Castellino, F. J., and Prorok, M. (2006) Cathepsin E is a specific marker of dysplasia in APC mouse intestine, Tumour Biol 27, 36-42.

Ullmann, R., Morbini, P., Halbwedl, I., Bongiovanni, M., Gogg-Kammerer, M., Papotti, M., Gabor, S., Renner, H., and Popper, H. H. (2004) Protein expression profiles in adenocarcinomas and squamous cell carcinomas of the lung generated using tissue microarrays, The Journal of pathology 203, 798-807.

Toshchakov, V., Jones, B. W., Perera, P. Y., Thomas, K., Cody, M. J., Zhang, S., Williams, B. R., Major, J., Hamilton, T. A., Fenton, M. J., and Vogel, S. N. (2002) TLR4, but not TLR2, mediates IFNbeta-induced STAT1alpha/beta-dependent gene expression in macrophages, Nature immunology 3,392-398.

Basu, S., Kang, T. J., Chen, W. H., Fenton, M. J., Baillie, L., Hibbs, S., and Cross, A. S. (2007) Role of Bacillus anthracis spore structures in macrophage cytokine responses, Infection and immunity 75, 2351-2358.

Kaplan EL, M. P. (1958) Nonparametric estimation from incomplete observations., Journal of the American Statistical Association 53, 457-481.

Thomas, K. E., Galligan, C. L., Deonarain Newman, R., Fish, E. N., and Vogel, S. N. (2006) Contribution of interferon (IFN)-beta to the murine macrophage response to the TLR4 agonist, lipopolysaccharide, J Biol Chem.

Tenenbaum, S. A., Lager, P. J., Carson, C. C., and Keene, J. D. (2002) Ribonomics: identifying mRNA subsets in mRNP complexes using antibodies to RNA-binding proteins and genomic arrays, Methods 26, 191-198.

Li, X. L., Andersen, J. B., Ezelle, H. J., Wilson, G. M., and Hassel, B. A. (2007) Post-transcriptional regulation of RNase-L expression is mediated by the 3′untranslated region of its mRNA, J Biol Chem.

Dong, B., Xu, L., Zhou, A., Hassel, B. A., Lee, X., Torrence, P. F., and Silverman, R. H. (1994) Intrinsic molecular activities of the interferon-induced 2-5A-dependent RNase, J Biol Chem 269, 14153-14158.

Le Roy, F., Salehzada, T., Bisbal, C., Dougherty, J. P., and Peltz, S. W. (2005) A newly discovered function for RNase L in regulating translation termination, Nat Struct Mol Biol 12, 505-512.

Dong, B., Niwa, M., Walter, P., and Silverman, R. H. (2001) Basis for regulated RNA cleavage by functional analysis of RNase L and Ire1p, Rna 7, 361-373.

Dong, B., and Silverman, R. H. (1997) A bipartite model of 2-5A-dependent RNase L, J Biol Chem 272, 22236-22242.

Carroll, S. S., Chen, E., Viscount, T., Geib, J., Sardana, M. K., Gehman, J., and Kuo, L. C. (1996) Cleavage of oligoribonucleotides by the 2′,5′-oligoadenylate-dependent ribonuclease L, J Biol Chem 271, 4988-4992.

Maitra, R. K., Li, G., Xiao, W., Dong, B., Torrence, P. F., and Silverman, R. H. (1995) Catalytic cleavage of an RNA target by 2-5A antisense and RNase L, J Biol Chem 270, 15071-15075.

Malathi, K., Paranjape, J. M., Ganapathi, R., and Silverman, R. H. (2004) HPC1/RNASEL mediates apoptosis of prostate cancer cells treated with 2′,5′-oligoadenylates, topoisomerase I inhibitors, and tumor necrosis factor-related apoptosis-inducing ligand, Cancer Res 64, 9144-9151.

Vasudevan, S., and Steitz, J. A. (2007) AU-rich-element-mediated upregulation of translation by FXR1 and Argonaute 2, Cell 128, 1105-1118.

Blasi, E., Radzioch, D., Merletti, L., and Varesio, L. (1989) Generation of macrophage cell line from fresh bone marrow cells with a myc/raf recombinant retrovirus, Cancer biochemistry biophysics 10, 303-317.

Amano, A., Nakagawa, I., and Yoshimori, T. (2006) Autophagy in innate immunity against intracellular bacteria, J Biochem (Tokyo) 140, 161-166.

Mizushima, N., Yamamoto, A., Matsui, M., Yoshimori, T., and Ohsumi, Y. (2004) In vivo analysis of autophagy in response to nutrient starvation using transgenic mice expressing a fluorescent autophagosome marker, Mol Biol Cell 15, 1101-1111.

Dengjel, J., Schoor, O., Fischer, R., Reich, M., Kraus, M., Muller, M., Kreymborg, K., Altenberend, F., Brandenburg, J., Kalbacher, H., Brock, R., Driessen, C., Rammensee, H. G., and Stevanovic, S. (2005) Autophagy promotes MHC class II presentation of peptides from intracellular source proteins, Proc Natl Acad Sci USA 102, 7922-7927.

Soreghan, B., Thomas, S. N., and Yang, A. J. (2003) Aberrant sphingomyelin/ceramide metabolicinduced neuronal endosomal/lysosomal dysfunction: potential pathological consequences in agerelated neurodegeneration, Advanced drug delivery reviews 55, 1515-1524.

Bosch, J. J., Thompson, J. A., Srivastava, M. K., Iheagwara, U. K., Murray, T. G., Lotem, M., Ksander, B. R., and Ostrand-Rosenberg, S. (2007) MHC class II-transduced tumor cells originating in the immune-privileged eye prime and boost CD4(+) T lymphocytes that cross-react with primary and metastatic uveal melanoma cells, Cancer Res 67, 4499-4506.

van Rooijen, N., Bakker, J., and Sanders, A. (1997) Transient suppression of macrophage functions by liposome-encapsulated drugs, Trends in biotechnology 15, 178-185.

Hummer, B. T., Li, X. L., and Hassel, B. A. (2001) Role for p53 in gene induction by double-stranded RNA, J Virol 75, 7774-7777.

Li, X. L., and Hassel, B. A. (2001) Involvement of proteasomes in gene induction by interferon and double-stranded RNA, Cytokine 14, 247-252.

Kopydlowski, K. M., Salkowski, C. A., Cody, M. J., van Rooijen, N., Major, J., Hamilton, T. A., and Vogel, S. N. (1999) Regulation of macrophage chemokine expression by lipopolysaccharide in vitro and in vivo, J Immunol 163, 1537-1544.

Salkowski, C. A., Detore, G., McNally, R., van Rooijen, N., and Vogel, S. N. (1997) Regulation of inducible nitric oxide synthase messenger RNA expression and nitric oxide production by lipopolysaccharide in vivo: the roles of macrophages, endogenous IFN-gamma, and TNF receptor-1-mediated signaling, J Immunol 158, 905-912.

Salkowski, C. A., Neta, R., Wynn, T. A., Strassmann, G., van Rooijen, N., and Vogel, S. N. (1995) Effect of liposome-mediated macrophage depletion on LPS-induced cytokine gene expression and radioprotection, J Immunol 155, 3168-3179.

Cailhier, J. F., Partolina, M., Vuthoori, S., Wu, S., Ko, K., Watson, S., Savill, J., Hughes, J., and Lang, R. A. (2005) Conditional macrophage ablation demonstrates that resident macrophages initiate acute peritoneal inflammation, J Immunol 174, 2336-2342.

Sledz, C. A., Holko, M., de Veer, M. J., Silverman, R. H., and Williams, B. R. (2003) Activation of the interferon system by short-interfering RNAs, Nat Cell Biol 5, 834-839.

Andersen, J. B., Li, X. L., Judge, C. S., Zhou, A., Jha, B. K., Shelby, S., Zhou, L., Silverman, R. H., and Hassel, B. A. (2006) Role of 2-5A-dependent RNase-L in senescence and longevity, Oncogene.

Xiang, Y., Wang, Z., Murakami, J., Plummer, S., Klein, E. A., Carpten, J. D., Trent, J. M., Isaacs, W. B., Casey, G., and Silverman, R. H. (2003) Effects of RNase L mutations associated with prostate cancer on apoptosis induced by 2′,5′-oligoadenylates, Cancer Res 63, 6795-6801.

Kokkinakis, D. M., Brickner, A. G., Kirkwood, J. M., Liu, X., Goldwasser, J. E., Kastrama, A., Sander, C., Bocangel, D., and Chada, S. (2006) Mitotic arrest, apoptosis, and sensitization to chemotherapy of melanomas by methionine deprivation stress, Mol Cancer Res 4, 575-589.

Lopez de Silanes, I., Zhan, M., Lal, A., Yang, X., and Gorospe, M. (2004) Identification of a target RNA motif for RNA-binding protein HuR, Proc Natl Acad Sci USA 101, 2987-2992.

Jiang, Q., Cross, A. S., Singh, I. S., Chen, T. T., Viscardi, R. M., and Hasday, J. D. (2000) Febrile core temperature is essential for optimal host defense in bacterial peritonitis, Infection and immunity 68, 1265-1270.

Kang, T. J., Fenton, M. J., Weiner, M. A., Hibbs, S., Basu, S., Baillie, L., and Cross, A. S. (2005) Murine macrophages kill the vegetative form of Bacillus anthracis, Infection and immunity 73, 7495-7501.

What is claimed is:

1. A method of treating a bacterial infection in a subject in need thereof, the method comprising administering an agent that increases the activity of RNase-L, said agent being a compound selected from the group consisting a compound represented by formula I, II, III, IV and pharmaceutical salts thereof, 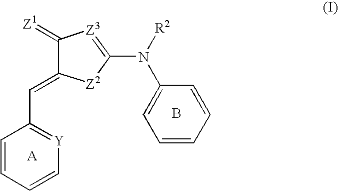
wherein,
Ring A and Ring B are optionally and independently substituted at any one or more substitutable ring carbon atoms;
Y is CH, N or N⁺—O⁻;
Z¹ and Z² are independently O or S;
Z³ is CR¹ or N;
R¹ is —H, —C(O)H, —C(O)R²⁰, —C(O)OR³⁰ or a C1-C5 alkyl group optionally substituted with one or more groups selected from halogen, hydroxyl, —OR²⁰, nitro, cyano, —C(O)H, —C(O)R²⁰, —C(O)OR³⁰, —OC(O)H and —OC(O)R²⁰ or R¹ is a group represented by the following structural formula: 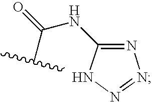
R² is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H or —OC(O)R²⁰;
each R²⁰ is independently C1-C3 alkyl or C1-C3 haloalkyl; and
R³⁰ is C1-C3 alkyl, C1-C3 haloalkyl or a group represented by a structural formula selected from: 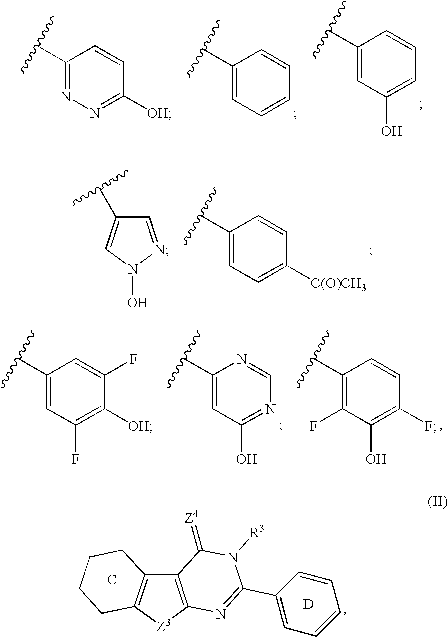
wherein,
Z³ and Z⁴ are independently O or S,
Ring C and Ring D are optionally and independently substituted at any one or more substitutable ring carbon atoms;
R³ is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰; and
each R²⁰ is independently C1-C3 alkyl or haloalkyl 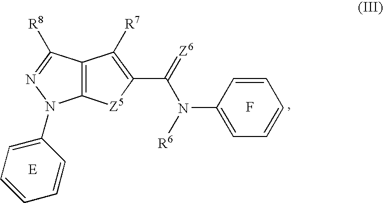
wherein,
Z⁵ and Z⁶ are independently O or S;
Ring E and Ring F are optionally and-independently substituted at any one or more substitutable ring carbon atoms;
R⁶ is —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R⁷ and R⁸ are independently —H, a C1-C5 alkyl group or a C1-C5 haloalkyl group; and
each R²⁰ is independently C1-C3 alkyl or haloalkyl 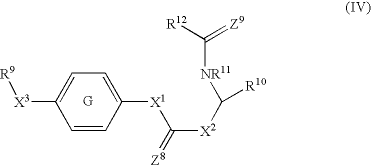
wherein,
X¹ and X² are independently CH₂, NH or O;
X³ is —O—C(O)—, —O—C(S)—, —S—C(O)—, —S—C(S)—, —C(O)—, C(S)—, —CH₂—, —CH(CH₃)—, —NHC(O)—, —C(O)NH—, —NHC(S)— or —C(S)NH—;
Z⁸ and Z⁹ are independently S or O;
Ring G is optionally substituted at any one or more substitutable ring carbon atoms;
R⁹ is a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R¹⁰ and R¹¹ are independently —H or a C1-C5 alkyl group optionally substituted with one or more groups selected from halogen, hydroxyl, —OR²⁰, nitro, cyano, —C(O)H, —C(O)R²⁰, —C(O)OR²⁰, —OC(O)H and —OC(O)R²⁰;
R¹² is —H; a C1-C5 alkyl group optionally substituted with one or more groups represented by R²¹; a monocyclic aromatic group optionally substituted at any one or more substitutable ring carbon atoms with a group represented by R²²; or a monocyclic C1-C3 aralkyl group optionally substituted at any one or more substitutable ring carbon atoms with R²³;
each R²⁰ is independently C1-C3 alkyl or C1-C3 haloalkyl;
each R²¹ is independently halogen, hydroxyl, —OR²⁰, nitro, cyano, —C(O)H, —C(O)R²⁰, —C(O)OR²⁰, —OC(O)H or —OC(O)R²⁰;
each R²² and R²³ is independently C1-C3 alkyl, C1-C3 haloalkyl, nitro, cyano, hydroxy, —OR²⁴, —C(O)H, —C(O)R²⁴, —C(O)OR²⁴, —OC(O)H, —OC(O)R²⁴ or C1-C3 alkyl substituted with hydroxyl, —OR²⁴, keto, —C(O)OR²⁴, —OC(O)H or —OC(O)R²⁴ and
R²⁴ is C1-C3 alkyl or C1-C3 haloalkyl.

2. The method of claim 1, wherein the compound is a compound represented by Formula I.

3. The method of claim 2, wherein the compound is selected from the group consisting of: 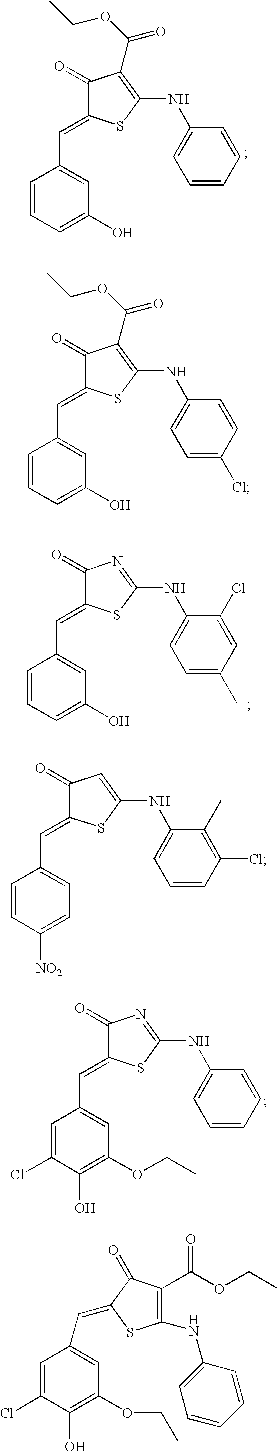
and pharmaceutically acceptable salts thereof.

4. The method of claim 1, wherein the compound is a compound represented by Formula II.

5. The method of claim 4, wherein the compound is selected from the group consisting of: 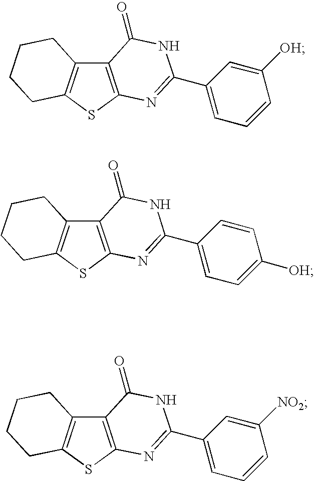
and pharmaceuticalyl acceptable salts thereof.

6. The method of claim 1, wherein the compound is a compound represented by Formula III.

7. The method of claim 6, wherein the compound is: 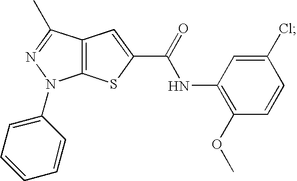
or a pharmaceutically acceptable salt thereof.

8. The method of claim 1, wherein the compound is a compound represented by Formula IV.

9. The method of claim 8, wherein the compound is: 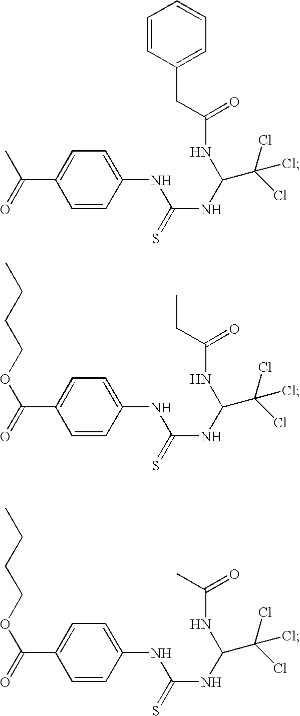
or a pharmaceutically acceptable salt thereof.

10. The method of claim 1, wherein the subject is not exhibiting symptoms of a viral infection prior to administration of the agent.

11. The method of claim 1 wherein the agent is co-administered with at least one additional therapy used to treat a bacterial infection.

12. A method of treating a bacterial infection in a subject in need thereof, the method comprising administering an agent that increases the activity of RNase-L, wherein said agent is a nucleic acid comprising cathepsinE mRNA or a 2′,5′-linked oligoadenylate (2-5A) mRNA.

13. The method of claim 12, wherein the subject is not exhibiting symptoms of a viral infection prior to administration of the agent.

14. The method of claim 12 wherein the agent is co-administered with at least one additional therapy used to treat a bacterial infection.

15. A method of treating a bacterial infection in a subject in need thereof, the method comprising administering an agent that increases the activity of RNase-L, wherein said agent increases the expression of RNase-L.

16. The method of claim 15 wherein the agent comprises a vector comprising a polynucleotide encoding RNase-L or encoding a functional part thereof.

17. The method of claim 15, wherein the subject is not exhibiting symptoms of a viral infection prior to administration of the agent.

18. The method of claim 15 wherein the agent is co-administered with at least one additional therapy used to treat a bacterial infection.
